(19) United States
(12) Patent Application Publication
(10) Pub. No.: US 2026/0345684 A1
(43) Pub. Date: Nov. 5, 2026

(54) B CELL INTERNALIZING BACTERIA WITH IMMUNE-ENHANCING EFFECTS AND PREPARING METHOD THEREOF

(71) Applicant: Seoul National University R&DB Foundation, Seoul (KR)

(72) Inventors: Youngnim Choi, Seoul (KR); Yeonkyeong Ko, Seoul (KR); Acharya Sabin, Seoul (KR)

(73) Assignee: Seoul National University R&DB Foundation, Seoul (KR)

(21) Appl. No.: 19/549,801

(22) Filed: Feb. 25, 2026

(30) Foreign Application Priority Data
Feb. 25, 2025 (KR) 10-2025-0024297
Feb. 24, 2026 (KR) 10-2026-0034287

(51) Int. Cl. A61K 35/17; A61K 9/00; A61K 40/13; A61K 40/24; A61K 40/45; A61P 31/04; C12N 5/0781

(52) U.S. Cl. CPC A61K 35/17; A61K 9/0019; A61K 40/13; A61K 40/24; A61K 40/45; A61P 31/04; C12N 5/0635; C12N 2502/70

(57) ABSTRACT

The present invention relates to B cells internalizing bacteria that exhibit an immune-enhancing effect and have therapeutic effects on bacterial infection or cancer. More specifically, the present invention relates to a method for preparing B cells internalizing bacteria, comprising co-culturing bacteria and isolated B cells; B cells internalizing bacteria prepared by the method; a cell therapeutic agent for preventing or treating cancer or bacterial infection, comprising the B cells; a pharmaceutical composition for preventing or treating cancer or bacterial infection, comprising the B cells; and a pharmaceutical composition for immune enhancement, comprising the B cells.

### SEQUENCE LISTING

The present application includes a Sequence Listing with file name SOP117084USsequencelisting.xml, which was created on Feb. 25, 2026, which has a size of 4,652 bytes, and which is hereby incorporated by reference in its entirety.

### TECHNICAL FIELD

This application claims priority to Korean Patent Application No. 10-2025-0024297, filed on Feb. 25, 2025, and Korean Patent Application No. 10-2026-0034287, filed on Feb. 24, 2026, and all contents disclosed in the specifications and drawings of the above applications are incorporated herein by reference.

The present invention relates to B cells internalizing bacteria that exhibit an immune-enhancing effect and have therapeutic effects on bacterial infection or cancer. More specifically, the present invention relates to a method for preparing B cells internalizing bacteria, comprising co-culturing bacteria and isolated B cells; B cells internalizing bacteria prepared by the method; a cell therapeutic agent for preventing or treating cancer or bacterial infection, comprising the B cells; a pharmaceutical composition for preventing or treating cancer or bacterial infection, comprising the B cells; and a pharmaceutical composition for immune enhancement, comprising the B cells.

### RELATED ART

Although their clinical manifestations differ, periodontitis, a chronic inflammatory disease, and Sjögren's disease (SjD), an autoimmune disease, share key immunopathological features, including persistent bacterial infection and marked B cell infiltration, and they often form tertiary lymphoid structures (TLSs). TLSs are ectopic lymphoid aggregates that are formed within non-lymphoid tissues under conditions of chronic inflammatory stress, such as persistent infection, autoimmunity or cancer.

TLSs are structurally similar to secondary lymphoid organs and comprise diverse B cell subsets, including naïve, memory, plasmablast, and atypical populations. However, they exhibit substantial variability in the degree of structural organization. Mature TLSs may form germinal center (GC), which constitute specialized microenvironments in which B cells refine antibody responses through interactions with T follicular helper (Tfh) cells. Nevertheless, fully developed GC are relatively rare within TLSs. Accordingly, the likelihood of antigen-specific interactions between B cells and T cells occurring within TLSs is significantly lower than that in classical secondary lymphoid tissues.

This raises a fundamental immunological paradox as to why, if TLSs represent inefficient sites for conventional B-T cell cooperation, memory B cells, which are typically generated through GC reactions with T cell help, are actively recruited to and abundantly present within peripheral inflamed tissues, such as the gingiva in periodontitis or the salivary glands in SjD.

Beyond their established role in antibody production, B cells can function as antigen-presenting cells (APCs) by capturing, processing, and presenting antigens via major histocompatibility complex class II (MHC II) molecules to CD4⁺ T cells while delivering the co-stimulatory signals necessary for T cell activation. Classical models, however, hold that B cells internalize antigens primarily through their B cell receptor (BCR), limiting their presentation to only the specific molecules they are programmed to recognize (non-patent document 1). For this reason, dendritic cells (DCs) are considered the principal APCs responsible for initiating T cell responses.

### PRIOR ART DOCUMENTS

### Non-Patent Document

(non-patent document 1) Lital N. Adler, et al., “The Other Function: Class II-Restricted Antigen Presentation by B Cells”, Front. Immunol., 8, 319

### SUMMARY

### Technical Problem

Under such background, the present inventors have proposed a hypothesis that B cells are recruited into peripheral tissues not merely for the purpose of antibody production, but also to function as antigen-presenting cells (APCs) that internalize bacteria and establish a local immune environment.

Accordingly, it was confirmed that human peripheral blood B cells efficiently internalize various oral bacteria in a BCR-independent manner, and thereafter upregulate antigen-presenting molecules and chemokines in vitro. In addition, using a mouse air pouch model, it was demonstrated that B cells internalizing bacteria recruit leukocytes to the site of injection and activate T cells in draining lymph nodes to induce the production of specific antibodies. Furthermore, the antigen-presenting capability was further confirmed through in vitro co-culture with T cells using both polyclonal populations and antigen-specific reporter systems, thereby completing the present invention.

Accordingly, an object of the present invention is to provide a method for preparing B cells internalizing bacteria, comprising co-culturing bacteria and isolated B cells.

Another object of the present invention is to provide B cells internalizing bacteria, prepared by the method.

Another object of the present invention is to provide B cells internalizing bacteria, wherein the B cells have internalized the bacteria independently of a BCR.

Still another object of the present invention is to provide a cell therapeutic agent for preventing or treating cancer or bacterial infection, comprising the B cells.

Still another object of the present invention is to provide a pharmaceutical composition for preventing or treating cancer or bacterial infection, comprising the B cells.

Furthermore, another object of the present invention is to provide a pharmaceutical composition for immune enhancement, comprising the B cells.

### Technical Solution

In order to solve the above-described problems, the present invention provides a method for preparing B cells internalizing bacteria, comprising co-culturing bacteria and isolated B cells.

In this case, the B cells may be isolated from human blood.

In this case, the bacteria and the B cells may be co-cultured at a multiplicity of infection (MOI) of 5 to 500.

In this case, the concentration of the B cells may be 1×10⁵/mL to 1×10⁶/mL.

In this case, the culture may be performed for 0.5 to 20 hours.

In this case, the bacteria may be internalized into the B cells independently of a B cell receptor (BCR).

In this case, the B cells internalizing bacteria may activate an immune response.

In this case, the B cells internalizing bacteria may have increased phagocytic activity, antigen-presenting capability, or chemokine secretion.

In addition, the present invention provides B cells internalizing bacteria prepared by the method.

In addition, the present invention provides B cells internalizing bacteria, wherein the B cells have internalized the bacteria independently of a BCR.

In addition, the present invention provides a cell therapeutic agent for preventing or treating cancer or bacterial infection, comprising the B cells.

In this case, the cancer may be at least one selected from the group consisting of breast cancer, ovarian cancer, uterine cancer, pancreatic cancer, lung cancer, gastric cancer, liver cancer, colorectal cancer, skin cancer, head or neck cancer, brain cancer, laryngeal cancer, prostate cancer, bladder cancer, esophageal cancer, thyroid cancer, kidney cancer, rectal cancer, leukemia and hematological malignancies.

Furthermore, the present invention provides a pharmaceutical composition for preventing or treating cancer or bacterial infection, comprising the B cells.

In this case, the cancer may be at least one selected from the group consisting of breast cancer, ovarian cancer, uterine cancer, pancreatic cancer, lung cancer, gastric cancer, liver cancer, colorectal cancer, skin cancer, head or neck cancer, brain cancer, laryngeal cancer, prostate cancer, bladder cancer, esophageal cancer, thyroid cancer, kidney cancer, rectal cancer, leukemia and hematological malignancies.

Furthermore, the present invention provides a pharmaceutical composition for immune enhancement, comprising the B cells.

### Advantageous Effects

The present inventors have identified a previously underappreciated immunological function of B cells as innate immunity-like antigen-presenting cells (APCs), which are capable of internalizing bacteria and initiating downstream immune responses independently of B cell receptor (BCR) engagement. In the present invention, it was confirmed that human and mouse B cells are capable of efficiently internalizing a broad range of bacterial species, including commensal bacteria and pathobionts associated with chronic inflammatory diseases, and that this process induces transcriptional and functional programs distinct from conventional BCR-mediated activation. B cells comprising internalized bacteria acquire APC-like properties, secrete chemokines, recruit leukocytes in vivo, and directly activate T cells through antigen presentation in vitro. Accordingly, it was confirmed that such B cells actively participate in early immune sensing and orchestration of immune responses beyond their conventional role in antibody production, thereby overall activating immune responses and exhibiting therapeutic effects on cancer or bacterial infection. Therefore, the B cells internalizing bacteria of the present invention have high utility value.

### BRIEF DESCRIPTION OF THE DRAWINGS

A of FIG. 1 shows representative images of immunohistochemical staining and in situ hybridization performed on tissue sections of periodontitis lesions and labial salivary glands from patients with Sjögren's syndrome, wherein CD20 (brown) labels B cells. B of FIG. 1 is a schematic diagram of an in vitro bacterial internalization assay using pHrodoRed-labeled bacteria and human B cells isolated from peripheral blood mononuclear cells. C of FIG. 1 shows representative flow cytometry plots of pHrodo-positive B cells after incubation with pHrodoRed-labeled bacteria. D of FIG. 1 shows quantitative analysis results of bacterial internalization efficiency among different bacterial species and donors, expressed as the percentage (%) of pHrodo-positive B cells (n=2-7 independent donors/species).

FIG. 2 shows results of evaluating the viability of human B cells after incubation with various pHrodoRed-labeled bacteria.

A of FIG. 3 shows representative confocal microscopy images obtained from assays using 2-3 independent donors (bacteria: red, nuclei: blue, actin: green). B of FIG. 3 shows scanning electron microscopy images illustrating bacterial uptake by B cells, wherein arrows indicate extracellular bacteria and arrowheads indicate internalized bacteria covered by membrane ruffles. C of FIG. 3 shows flow cytometry results of B cells derived from two donors incubated with whole oral wash samples (OS1-OS3). D of FIG. 3 shows the number of identified taxa in oral wash samples and intra-B-cell bacterial communities, determined by 16S rRNA sequencing. E of FIG. 3 shows a multidimensional scaling (MDS) plot of bacterial communities based on Bray-Curtis dissimilarity.

FIG. 4 shows results of evaluating an internalization index for various bacteria, defined as a ratio of relative abundance within B cells to relative abundance in the corresponding oral wash sample.

A of FIG. 5 shows the percentage of Pm-containing cells in each sample. B of FIG. 5 shows a sorting strategy for isolating Pm⁺ and Pm⁻ B cell populations. C of FIG. 5 shows results of principal coordinate analysis (PCoA) illustrating sample relationships based on global transcriptional profiles. D of FIG. 5 shows a heatmap representing the expression of subset-defining B-cell marker genes. E of FIG. 5 shows the number of differentially expressed genes (DEGs) used for Gene Ontology analysis. F and G of FIG. 5 show terms enriched among DEGs from Pm⁺ vs. NI and Pm⁺ vs. Pm⁻ comparison.

A of FIG. 6 shows a schematic overview of the experimental design for single-cell RNA sequencing. B of FIG. 6 shows the proportion of Pm-internalizing cells (Pm⁺) and overall cell viability for each sample determined by flow cytometry. C of FIG. 6 shows a UMAP (Uniform Manifold Approximation and Projection) plot representing cell distribution across identified clusters (C0-C9), which was analyzed in integration with previously published and curated immune cell data (shown in gray; see FIG. 7), and the bar graph shows the proportion of cells in each cluster for NI and Pm-infected samples.

FIG. 7 is a UMAP plot showing the distribution of curated, previously published immune cell datasets integrated with data from the current study.

FIG. 8 is a heatmap displaying the expression of subset-specific marker genes across identified clusters.

A of FIG. 9 shows results of pseudotime trajectory analysis. B and C of FIG. B and C show the number of differentially expressed genes and results of Gene Ontology enrichment analysis between the NI and Pm-infected B cells. D of FIG. 9 shows the percentage of Pm⁺ cells identified in each cluster using the SAHMI (single-cell analysis of host-microbiome interactions) algorithm. E and F of FIG. 9 show the number of DEGs and results of GO enrichment analysis identified in the comparison between Pm⁺ cells and NI.

A of FIG. 10 shows a heatmap comparing chemokine gene expression between Pm vs. NI and Pm⁺ vs. NI comparisons. B of FIG. 10 shows predicted interactions between upregulated B cell-derived chemokines and target leukocytes.

A of FIG. 11 shows a schematic overview of the experimental design, wherein human B cells were infected with Pm, Ec, or Se for 72 hours, treated with gentamicin 1 hour post-infection, and then subjected to phenotypic and functional analyses. B of FIG. 11 shows results of quantifying bacterial internalization efficiency and B cell viability after 72 hours of culture by flow cytometry in samples subjected to mass cytometry.

A of FIG. 12 shows a t-SNE plot of mass cytometry data analyzed via FlowSOM (C0-C9) and the corresponding cell counts per cluster across experimental groups. B of FIG. 12 shows the mean intensity of surface markers in C1 and C2 across experimental groups.

A of FIG. 13 shows a schematic overview of gating strategy for defining live cells in mass cytometry data. B of FIG. 13 shows the proportion of marker-positive cells among live cells in mass cytometry. C of FIG. 13 shows a gating strategy for identifying naïve (IgD⁺CD27⁻), memory (CD27⁺), and double negative (DN; IgD-CD27⁻) B cell subsets using flow cytometry.

A of FIG. 14 shows results of flow cytometric analysis of the distribution of each B cell subset (left; gating strategy shown in C of FIG. 13) and the proportion of pHrodo-positive cells within each subset (right) at 1 hour and 72 hours in the non-infected group (NI), Pm⁻, Ec⁻, or Se⁻ infected B cells. B of FIG. 14 shows antibody concentrations in culture supernatants measured by ELISA. C of FIG. 14 shows results of B cell proliferation assessed based on the proportion of CFSE^{lo} cells. FIG. D of 14 shows results of an antibiotic protection assay for evaluating the survival of intracellular bacteria after 72 hours of culture (CFU per whole culture). E of FIG. 14 shows a dot blot image of a cytokine array performed on pooled supernatants. F of FIG. 14 shows signal intensities of the dots shown in E. F of FIG. 14 shows results of ELISA measurements of IL-16, IL-18, and TNF-α levels in individual culture supernatants.

A and B of FIG. 15 show the proportion of bacteria-containing cells and phospho-Syk⁺ cells in human (A) and mouse primary B cells (B) after incubation with α-IgM or pHrodo Red-labeled bacteria. C of FIG. 15 shows the experimental design for bulk RNA sequencing, in which mouse primary B cells were incubated with α-IgM or Ec for 16 hours, and gentamicin was added 1 hour after incubation. D of FIG. 15 shows a principal coordinates analysis (PCoA) plot based on transcriptomic profiles. E of FIG. 15 shows the number of differentially expressed genes (DEGs). F to H of FIG. 15 show Gene Ontology enrichment analysis results for DEGs: Ec and α-IgM vs. NI comparison (F), α-IgM vs. NI comparison (G), and Ec vs. NI comparison (H).

A of FIG. 16 shows a schematic diagram of an air pouch model in C57BL/6 mice. B of FIG. 16 shows the proportion of bacteria-containing cells among injected CD45.1+ B cells. C of FIG. 16 shows flow cytometry plots and count of B cells (CD3⁻CD19⁺) and T cells (CD3⁺CD19⁻) in lavage fluid. D of FIG. 16 shows flow cytometry plots defining neutrophils (CD11b⁺Ly6G⁺) and monocytes (CD11b⁺Ly6G⁻) gated from CD3⁻CD19⁻ cells, and their respective counts in lavage fluid. E of FIG. 16 shows flow cytometry plots and counts of donor CD45.1⁺ B cells in the lavage fluid. F of FIG. 16 shows flow cytometry plots and counts of pHrodoRed-positive CD45.1⁺ (donor) and CD45.1⁻ (host) B cells in the lavage fluid.

A of FIG. 17 shows the proportion of pHrodoRed-labeled bacteria-containing cells among recruited host leukocytes. B of FIG. 17 shows a representative histogram of proliferating CD45.1⁺ B cells and the frequency in the lavage fluid. C of FIG. 17 shows a representative flow cytometry plot of Annexin V⁺CD45.1⁺ B cells along with their frequency and cell counts in the lavage fluid. D of FIG. 17 shows the Counts of B cells, T cells, neutrophils, and monocytes examined at 2 hours and 1 day post-injection (1 dpi).

FIG. 18 shows flow cytometry plots and counts of CD45.1⁺ and CD45.1⁻ B cells in draining lymph nodes (D.LNs), non-draining lymph nodes (ND.LNs), and spleen (SPL).

A of FIG. 19 shows the frequency of CD45.1⁺ cells among CD3⁻CD19⁺ B cells in lymph nodes and spleen. B of FIG. 19 shows a representative histogram of proliferating CD45.1⁺ B cells and the frequency of the cells in lymph nodes and spleen. C of FIG. 19C shows a representative flow cytometry plot of pHrodoRed-labeled bacteria-containing B cells and the number of pHrodo⁺ CD45.1⁺ and CD45.1⁻ B cells.

A and B of FIG. 20 show representative plots and frequency/MFI of CD80, CD86, and MHC II on B cell subsets. C and D of FIG. 20 show the total counts and frequency of CD44^{hi} activation within CD4⁺ and CD4⁻ T cell populations.

A to C of FIG. 21 show representative flow cytometry plots of IFNγ⁺ (D), IL-17⁺ (E), and FoxP3⁺ (F) cells, along with their frequency among CD4⁺CD44^{hi} and CD4-CD44^{hi} T cells. D of FIG. 21 shows the total CFU counts measured in cell lysates from lavage fluid and draining lymph nodes.

A of FIG. 22 shows the experimental design for the 14-dpi analysis, and B of FIG. 22 shows the proportion of bacteria-containing cells among injected CD45.1⁺ B cells.

A of FIG. 23 shows representative flow cytometry plots defining CD3⁺ T cells (green) and CD19⁺ B cells (red) among live cells, and the number of CD19⁺ cells in lymph nodes and spleen. B of FIG. 23 shows representative flow cytometry plots defining CD3⁺CD4⁺ T cells and CD3⁺CD4⁻ T cells, with corresponding cell counts.

A of FIG. 24 shows flow cytometry plots and counts of antibody-secreting cells (ASCs) in draining lymph nodes, non-draining lymph nodes, and spleen. B and C of FIG. 24 show the serum concentrations of total, Ec-specific and Se-specific IgG (D), and IgM (E).

FIG. 25 shows the concentrations of Pm⁻ and Ss⁻ specific IgM measured in sera using ELISA.

A of FIG. 26 shows a schematic for assessing the capacity of B cells and DCs to activate T cells in vitro. B of FIG. 26 shows a gating strategy for the flow cytometric analysis cytokine-producing T cells. C and D of FIG. 26 show the proportion of IFN-7⁺ or IL-17⁺ cells among CD44^{hi}CD4⁺ T cells and cytokine concentrations after coculture with bacteria-infected B cells (H) or DCs (I).

A of FIG. 27 shows the sequence information of a synthesized 747-bp fragment (containing the Fim promoter, FimC N-terminal, His-tag, and OT-II peptide) and the 1257-bp FimC C-terminal DNA fragment amplified by PCR. B of FIG. 27 shows a map of the pTCOW OTII FimC plasmid into which the two DNA fragments shown in A are cloned. C of FIG. 27 shows Western blot analysis results: lysates from pTCOW-Pg (empty vector control) and OTII-Pg immunoblotted with anti-His tag antibodies.

FIG. 28 shows activation of OT-II T cells co-cultured with bacteria-infected B cells.

### DETAILED DESCRIPTION

Hereinafter, the present invention will be described in more detail.

Meanwhile, each of the descriptions and embodiments disclosed herein may be applied to other descriptions and embodiments. That is, all combinations of various elements disclosed herein fall within the scope of the present invention. In addition, the scope of the present invention is not to be construed as being limited by the specific descriptions set forth below.

Further, those skilled in the art may recognize or ascertain many equivalents to specific aspects of the present invention described in this application using only routine experimentation. Such equivalents are also intended to be encompassed within the scope of the present invention.

The present inventors have completed the present invention by confirming that B cells that internalize bacteria in a BCR-independent manner activate overall immune responses through increased chemokine secretion and antigen-presenting capacity.

In a specific embodiment of the present invention, it was confirmed that human B cells can internalize bacteria in vitro (FIGS. 1 to 4).

Accordingly, a first aspect of the present invention relates to a method for preparing B cells internalizing bacteria, comprising co-culturing bacteria and isolated B cells.

As used herein, the term “internalization” refers to a state in which bacteria pass through the cell membrane of B cells and enter the intracellular space of the B cells, for example, the cytoplasm, endosome, phagosome, or other intracellular organelles, and exist within the cell. Such internalization may occur via active or passive mechanisms, for example, through endocytosis, phagocytosis, or other known intracellular uptake mechanisms.

In the present invention, the bacteria may be Staphylococcus aureus, Staphylococcus epidermidis, Streptococcus oralis, Streptococcus salivarius, Rothia mucilaginosa, Escherichia coli, Porphyromonas gingivalis, Prevotella melaninogenica, Veillonella dispar, Phyllobacterium myrsinacearum, Sphingomonas melonis, Sphingomonas paucimobilis, Methylorubrum zatmanii, or Alcaligenes faecalis, but are not limited thereto.

In the present invention, the bacteria may be live bacteria or dead bacteria (e.g., heat-killed, formalin-fixed, etc.).

In the present invention, the B cells may be isolated from mammals, for example, humans, mice, rats, rabbits, dogs, pigs, or monkeys, and are preferably isolated from blood-derived samples, for example, human blood such as peripheral blood, cord blood, or peripheral blood mononuclear cells separated therefrom.

In the present invention, the bacteria and the B cells may be co-cultured at a multiplicity of infection (MOI) of 5 to 500. Preferably, the MOI is 10 to 500, and most preferably 10 to 100.

As used herein, the term “MOI (multiplicity of infection)” refers to the ratio of the number of microorganisms, for example, bacteria, to the number of target cells, and indicates the average number of microorganisms applied per cell. The MOI is defined as the ratio of the total number of microorganisms to the total number of cells, and may be expressed, for example, as MOI 1, MOI 10, MOI 100, and the like. The microorganisms may be treated so as to contact, be co-cultured with, or be internalized into the cells.

In the present invention, the concentration of the B cells may be 1×10⁵/mL to 1×10⁶/mL. Preferably, it is 1×10⁵/mL to 5×10⁵/mL, and most preferably 2.5×10⁵/mL.

In the present invention, the culture may be performed for 0.5 to 100 hours. Preferably, the culture is performed for 0.5 to 50 hours, more preferably 0.5 to 25 hours, and most preferably 0.5 to 16 hours.

In a specific embodiment of the present invention, it was confirmed that bacterial internalization induces a broad transcriptional program independent of the B cell receptor (BCR), thereby demonstrating that the bacteria are internalized in a BCR-independent manner (FIG. 15).

Accordingly, in the present invention, the bacteria may be internalized into the B cells independently of a B cell receptor.

In a specific embodiment of the present invention, it was confirmed that internalization of the bacteria induces broad and subset-specific transcriptional reprogramming in B cells, including increased endocytic activity, enhanced antigen-presenting function, and increased chemokine production (FIGS. 5 to 10).

In another specific embodiment of the present invention, it was confirmed that B cells internalizing bacteria exhibit an antigen-presenting cell-like phenotype and secrete inflammatory chemokines (FIGS. 11 to 14).

Accordingly, in the present invention, the B cells internalizing bacteria may activate an immune response.

In the present invention, the B cells internalizing bacteria may exhibit increased phagocytic activity, antigen-presenting ability, or chemokine secretion.

As used herein, the term “phagocytic activity” refers to the ability of a cell to capture, ingest, and internalize bacteria, particles, or other external substances into the cell through invagination of the cell membrane. The term “antigen-presenting ability” means the ability of a cell to present, on the cell surface, in a form bound to major histocompatibility complex (MHC) molecules, for example MHC class I or MHC class II molecules, so as to enable recognition by or activation of T cells. The term “chemokine secretion” refers to the ability of a cell to produce and release chemokines, which are signaling proteins that induce migration, chemotaxis, or activation of immune cells.

A second aspect of the present invention relates to B cells internalizing bacteria prepared by the method.

The constitution and effects of the B cells internalizing bacteria are the same as those described in the first aspect, and thus the description thereof is omitted.

A third aspect of the present invention relates to B cells internalizing bacteria, wherein the B cells have internalized the bacteria independent of a BCR.

The constitution and effects of the B cells internalizing bacteria are the same as those described in the first aspect, and thus the description thereof is omitted.

In the present invention, it was confirmed that the B cells internalizing bacteria effectively activate immune cells to enhance overall immune responses. In particular, it was confirmed that the B cells internalizing bacteria increase the production and secretion of bacteria-specific antibodies, for example IgG, thereby enhancing antibacterial immune responses, and thus exhibit therapeutic effects against bacterial infections. In addition, the increase in immune cell activation and antibody production induced by the B cells of the present invention may also enhance immune responses against tumor cells, and the increased antibodies and immune activation may promote anti-tumor immune responses in the tumor microenvironment.

Accordingly, a fourth aspect of the present invention relates to a cell therapeutic agent for preventing or treating cancer or bacterial infection, comprising the B cells.

In the present invention, the cell therapeutic agent may be an immune cell therapeutic agent.

As used herein, the term “cell therapeutic agent” refers to cells administered to a subject for therapeutic purposes, wherein the cells may provide therapeutic effects through immune regulation, antigen presentation, antibody production, tissue protection, tissue regeneration, or other biological functions. For this purpose, the cells may undergo a series of processes such as ex vivo expansion, selection, or other methods for altering the biological characteristics of the cells, using living autologous or allogeneic cells, and are pharmaceutical products used for the purposes of treatment, diagnosis, and prevention (Regulation on Approval and Review of Biological Products, Korea Food and Drug Administration Notice).

As used herein, the term “immune cell therapeutic agent” refers to a therapeutic agent comprising cells capable of activating or regulating immune responses, wherein the cells may enhance immune responses through antigen presentation, antibody production, or immune cell activation functions. In one embodiment, the immune cell therapeutic agent may comprise B cells internalizing bacteria, and the B cells may exert therapeutic effects by inducing immune cell activation and antibody production. The immune cell therapeutic agent may further comprise other immunotherapeutic agents.

In the present invention, the cancer may be any one selected from the group consisting of breast cancer, ovarian cancer, uterine cancer, pancreatic cancer, lung cancer, gastric cancer, liver cancer, colorectal cancer, skin cancer, head or neck cancer, brain cancer, laryngeal cancer, prostate cancer, bladder cancer, esophageal cancer, thyroid cancer, kidney cancer, rectal cancer, leukemia, and hematological cancer.

In the present invention, the bacterial infection (bacterial infectious disease) refers to any disease or condition caused by or associated with the presence, proliferation, or activity of bacteria, and may include, but is not limited to, tissue infection, blood infection, mucosal infection, or organ infection. The bacterial infection may be caused by Staphylococcus aureus, Staphylococcus epidermidis, Streptococcus oralis, Streptococcus salivarius, Rothia mucilaginosa, Escherichia coli, Porphyromonas gingivalis, Prevotella melaninogenica, Veillonella dispar, Phyllobacterium myrsinacearum, Sphingomonas melonis, Sphingomonas paucimobilis, Methylorubrum zatmanii, or Alcaligenes faecalis, but is not limited thereto.

A fifth aspect of the present invention relates to a pharmaceutical composition for preventing or treating cancer or bacterial infection, comprising the B cells.

The constitution and effects of the composition are the same as those described in the fourth aspect, and thus the description thereof is omitted.

As used herein, the term “prevention” refers to any act of suppressing or delaying the onset of cancer or bacterial infection or related diseases by the B cells of the present invention.

As used herein, the term “treatment” refers to any act of improving or beneficially changing parameters associated with cancer or bacterial infection or related diseases, for example, the degree of symptoms, by the B cells of the present invention.

The cell therapeutic agent or pharmaceutical composition of the present invention may be in various parenteral formulations.

When formulating the cell therapeutic agent or composition, it may be prepared using one or more buffers (e.g., saline or PBS), antioxidants, bacteriostatic agents, chelating agents (e.g., EDTA or glutathione), fillers, bulking agents, binders, adjuvants (e.g., aluminum hydroxide), suspending agents, thickening agents, wetting agents, disintegrants, surfactants, diluents, or excipients.

The cell therapeutic agent or composition of the present invention may be administered parenterally, and for parenteral administration, it may be formulated according to methods known in the art, such as injections for intraperitoneal, rectal, intravenous, intramuscular, or subcutaneous administration.

In the case of injections, they must be sterile and protected from contamination by microorganisms such as bacteria and fungi, and may comprise the living B cells of the present invention. Examples of suitable carriers for injections include, but are not limited to, solvents or dispersion media including water, ethanol, polyols (e.g., glycerol, propylene glycol, and liquid polyethylene glycol), mixtures thereof, and/or vegetable oils. More preferably, suitable carriers may include Hanks' solution, Ringer's solution, PBS (phosphate-buffered saline) comprising triethanolamine, sterile water for injection, isotonic solutions such as 10% ethanol, 40% propylene glycol, and 5% dextrose. In order to protect the injections from microbial contamination, various antibacterial and antifungal agents such as parabens, chlorobutanol, phenol, sorbic acid, and thimerosal may be further included. In addition, in most cases, the injections may further comprise isotonic agents such as sugars or sodium chloride.

The cell therapeutic agent or composition of the present invention is administered in a pharmaceutically effective amount. The pharmaceutically effective amount means an amount sufficient to treat a disease at a reasonable benefit/risk ratio applicable to medical treatment, and the effective dosage level may be determined depending on factors including the type and severity of the disease of the patient, drug activity, sensitivity to the drug, time of administration, route of administration and rate of excretion, duration of treatment, concomitant drugs, and other factors well known in the medical field. The composition of the present invention may be administered as a single therapeutic agent or in combination with other therapeutic agents, and may be administered sequentially or simultaneously with conventional therapeutic agents, and may be administered in a single dose or in multiple doses. That is, the total effective amount of the composition of the present invention may be administered to a patient as a single dose or may be administered by a fractionated treatment protocol in which multiple doses are administered over a long period of time. Considering all of the above factors, it is important to administer an amount that can achieve maximum effect with a minimum amount without side effects, and such an amount can be easily determined by those skilled in the art.

The dosage of the cell therapeutic agent or pharmaceutical composition of the present invention may vary depending on the body weight, age, sex, health condition, diet, administration time, administration method, excretion rate, and severity of the disease of the patient.

The cell therapeutic agent or composition of the present invention may be used alone or in combination with methods using surgery, radiotherapy, hormone therapy, chemotherapy, and biological response modifiers.

In a specific embodiment of the present invention, it was confirmed that the B cells internalizing bacteria can induce local innate and adaptive immune responses, and further systemic immune responses (FIGS. 16 to 28).

Accordingly, a sixth aspect of the present invention relates to a composition for immune enhancement comprising the B cells of claim 9.

In the present invention, the composition may be a pharmaceutical composition for immune enhancement.

As used herein, the term “immune enhancement” may mean regulating an immune response occurring in the body in a beneficial direction. For example, immune enhancement may suppress an immune response when an excessive inflammatory response occurs, and conversely, may induce an inflammatory response to treat infection when infection occurs. In the present invention, immune enhancement may specifically mean enhancing immune function.

In addition, a seventh aspect of the present invention relates to a method for preventing or treating cancer or bacterial infection, comprising administering the B cells to a subject in need thereof.

In the present invention, the subject may be a subject at risk of developing or having developed cancer or a bacterial infection, and may include, but is not limited to, any animal (e.g., human, horse, pig, rabbit, dog, sheep, goat, non-human primate, cow, cat, guinea pig, or rodent). Such terms do not denote a particular age or sex. Accordingly, it is intended to include female/male, adult and neonatal subjects, as well as fetuses.

In addition, an eighth aspect of the present invention relates to a method for inducing an immune response, comprising administering the B cells to a subject in need thereof.

In the present invention, the subject may be a healthy subject (normal subject), or a subject at risk of or having developed a disease requiring induction of an immune response, that is, a subject in which induction of an immune response is required for preventive or therapeutic purposes. The subject may include any subject for the purpose of prophylactic enhancement of immune response, enhancement of immune function, or prevention and treatment of disease. The subject may include, but is not limited to, any animal (e.g., human, horse, pig, rabbit, dog, sheep, goat, non-human primate, cow, cat, guinea pig, or rodent). Such terms do not denote a particular age or sex. Accordingly, it is intended to include female/male, adult and neonatal subjects, as well as fetuses.

In the present invention, the immune response includes both innate immune responses and adaptive immune responses. The immune response includes, but is not limited to, activation, proliferation, differentiation, and migration of immune cells, secretion of cytokines and chemokines, antigen presentation, T cell activation, antibody production, and induction of cytotoxic responses.

Hereinafter, the present invention will be described in more detail by way of the following examples. However, the following examples are provided for illustrative purposes only, and the scope of the present invention is not limited thereto.

### Example 1

### 1. Materials and Methods

### 1-1. Immunohistochemistry and In Situ Hybridization

Human tissue sections were obtained from the Department of Oral Pathology at Seoul National University Dental Hospital, with approval from the Institutional Review Board (IRB) of Seoul National University School of Dentistry (IRB no. S-D20230023). Paraffin-embedded gingival tissues from periodontitis patients (n=5) and labial salivary glands from SjD patients (n=8) underwent immunohistochemistry (IHC) for CD20, followed by in situ hybridization (ISH) for bacterial 16S rRNA. After deparaffinization, antigen retrieval (citrate buffer, pH 6.0), and blocking, sections were incubated with anti-human CD20 (1:50, clone L26, eBioscience, no. 14-0202-82), followed by goat anti-mouse IgG2a-HRP (1:100, SouthernBiotech, no. 1081-05), and visualized with DAB. ISH involved sequential pre-treatment, hybridization with a DIG-labeled universal 16S rRNA probe, and detection via anti-DIG-AP with NBT/BCIP. Negative controls used excess unlabeled probe. Sections were counterstained with 0.5% methyl green, mounted, and examined microscopically.

1-2. Identification of Bacteria from the Salivary Gland Proteomics Public Dataset

The public salivary gland proteome dataset (PRIDE accession no. PXD016231), reporting data from 19 primary SjD patients and 20 non-SjD controls, was retrieved using the PRIDE API and ProteomeXchange submission interface. Raw data were analyzed using Mascot (v2.5.1) against in-house composite human and bacterial protein databases. The human database included UniProt-reviewed proteins plus 260 common contaminants, and the bacterial database was built from eHOMD sequences refined by BLAST against UniProtKB/Swiss-Prot. Identifications were validated in Scaffold (v5.2.0) with >2 unique peptides per protein, 0.1% peptide-level false discovery rate (FDR), and 1% protein-level FDR. Results are presented in Table 1 below.

TABLE 1

| | Detection cases: % (n) from | | | | | | |
|---|---|---|---|---|---|---|---|
| | Total | | Non-SjD | | SjD | | |
| Bacterial species | (n = 39) | | (n = 20) | | (n = 19) | | ^{a}p |
| Streptococcus oralis | 66.7 | (26) | 85 | (17) | 47.4 | (9) | 0.02 |
| ATCC 9811 | | | | | | | |
| Mogibacterium timidum | 30.8 | (12) | 30 | (6) | 31.6 | (6) | 1 |
| ATCC 33093 | | | | | | | |
| Achromobacter | 23.1 | (9) | 25 | (5) | 21.1 | (4) | 1 |
| xylosoxidans A8 | | | | | | | |
| Staphylococcus | 20.5 | (8) | 20 | (4) | 21.1 | (4) | 1 |
| epidermidis W23144 | | | | | | | |
| Pseudomonas fluorescens | 10.3 | (4) | 15 | (3) | 5.3 | (1) | 0.6 |
| Pf-5 | | | | | | | |
| Cronobacter sakazakii | 10.3 | (4) | 10 | (2) | 10.5 | (2) | 1 |
| ATCC BAA-894 | | | | | | | |
| Finegoldia magna | 7.7 | (3) | 5 | (1) | 10.5 | (2) | 0.6 |
| BVS033A4 | | | | | | | |
| Mycoplasma genitalium | 7.7 | (3) | 0 | (0) | 15.8 | (3) | 0.1 |
| G-37 ATCC 33530 | | | | | | | |
| Pseudomonas | 7.7 | (3) | 10 | (2) | 5.3 | (1) | 1 |
| pseudoalcaligenes KF707 | | | | | | | |
| Bacillus anthracis A0248 | 5.1 | (2) | 5 | (1) | 5.3 | (1) | 1 |
| Cardiobacterium hominis | 5.1 | (2) | 10 | (2) | 0 | (0) | 0.5 |
| ATCC 15826 | | | | | | | |
| Lactobacillus | 5.1 | (2) | 5 | (1) | 10.5 | (2) | 1 |

acidophilus NCFM

^{a}By Chi-square test

1-3. pHrodoRed Labeling of Bacteria

S. salivarius KCTC 3960 (Ss), Rothia mucilaginosa KCTC 19862 (Rm), Prevotella melaninogenica KCTC 5457 (Pm), and Staphylococcus epidermidis KCTC 1917 (Se) were purchased from the Korean Collection for Type Cultures (KCTC). Veillonella dispar ATCC 17748 (Vd), Porphyromonas gingivalis ATCC 33277 (Pg), Staphylococcus aureus ATCC 29213 (Sa), and Streptococcus oralis ATCC 6249 (So) were purchased from the American Type Culture Collection (ATCC). Escherichia coli strain 7.2 (Ec) was clinically isolated (37).

Ss, Rm, Sa, So, and Se were cultured aerobically at 37 °C in brain heart infusion (BHI) broth. Ec was grown in LB broth at 37 °C aerobically. Pm and Vd were cultured anaerobically (10% CO₂, 5% H₂, 85% N₂) at 37 °C in KCTC medium No. 282 (Modified PYG) and ATCC medium 188, respectively. Pg was cultured anaerobically at 37 °C in BHI supplemented with κ g/ml hemin and 5 g/ml vitamin K.

Bacteria were harvested during logarithmic growth phase, washed twice with DPBS, and incubated with 20 M pHrodo™ Red succinimidyl ester (Invitrogen no. P36600) for 30 min at room temperature, following the manufacturer's instructions. Labeled bacteria were washed once with DPBS, followed by a wash with 20% methanol in DPBS. The cells were then resuspended in DPBS at a concentration determined by optical density (OD) measurements.

### 1-4. Construction of OTII-Pg

A synthetic gene construct (747 bp) consisting of the Pg Fim promoter, the N-terminal 132 bp of fimC, a His-tag sequence, and the OT-II epitope was synthesized (Genewiz Inc., Burlington, USA), and the C-terminal region of fimC (1257 bp) was amplified by PCR (A of FIG. 27). The synthesized gene construct and amplified fragment were assembled into the pTCOW plasmid linearized with SmaI using the HiFi DNA Assembly Cloning Kit (New England BioLabs, no. E5520S), following the manufacturer's instructions (B of FIG. 27). The resulting plasmid was transformed into E. coli DH5α and verified by Sanger sequencing. Subsequently, the plasmid was transformed into E. coli S17-1 and conjugated into Pg. Expression of the OT-II peptide was confirmed by western blotting using anti-His-tag antibodies (BioLegend, no. 652502) (C of FIG. 27).

1-5. Preparation of Primary B Cells and Incubation with Bacteria

The use of human materials was approved by the IRB of Seoul National University School of Dentistry (IRB no. S-D20150007). Human B cells were obtained from peripheral blood supplied by the Korean Red Cross; no donor information was provided. Peripheral blood mononuclear cells (PBMCs) were isolated using Ficoll-Paque PLUS (GE Healthcare, no. 17-1440-03), and B cells were purified using the EasySep™ Human B Cell Isolation Kit (STEMCELL Technologies, no. 17954).

Mouse B cells were isolated from spleens. After mechanical dissociation, red blood cells were lysed using RBC lysing buffer (Sigma-Aldrich, no. R7757-100). B cells were then purified using the EasySep™ Mouse Pan B Cell Isolation Kit (STEMCELL Technologies, no. 19844A).

Isolated B cells (2.5×10⁵/ml) were incubated with pHrodoRed-labeled bacteria at an MOI of 10 or 100 in RPMI 1640 medium (HyClone, no. SH30027.01) supplemented with 10% FBS and 50 μM 2-mercaptoethanol (Sigma-Aldrich, no. S7215) at 37 °C in a 5% CO₂ incubator. To prevent bacterial overgrowth, 50 μg/ml gentamicin (Gibco, no. 15750-060) was added 1 h post-infection. For human B cell cultures extending ≥72 h, 10% human serum (Sigma-Aldrich, no. H3667) was substituted for FBS to improve cell survival, except for specific functional assays.

### 1-6. Confocal Microscopy

Human B cells (2.5×10⁵/well) were seeded onto gelatin-coated confocal-grade imaging plates and incubated with pHrodoRed-labeled bacteria at an MOI of 100 for 1 h at 37 °C After washing three times with DPBS to remove unbound bacteria, cells were fixed and stained with Alexa Fluor 647-conjugated phalloidin (1:100, Invitrogen, no. A22287) and DAPI (1:1000, Invitrogen, no. D3571). Confocal images were acquired using a ZEISS LSM 980 microscope equipped with Airyscan 2 (Carl Zeiss).

### 1-7. Scanning Electron Microscopy

Cells (2.5×10⁵) infected with pHrodoRed-labeled bacteria at an MOI of 100 for 1 h were fixed with 2.5% glutaraldehyde for 2 h at 4 °C, post-fixed with 1% osmium tetroxide on ice for 1 h, dehydrated, and chemically dried. Samples were coated with platinum using a Q150TS sputter coater (Quorum Technologies) and imaged with an Apreo S microscope (Thermo Fisher Scientific) at 10 kV, 0.10 nA.

### 1-8. Microbiota Analysis of Oral Wash Samples and B Cell-Internalized Bacteria

Human oral wash samples were collected with approval from the IRB of the Seoul National University School of Dentistry (IRB no. S-D20230009), and written informed consent was obtained from all participants. Samples were obtained from three healthy volunteers (two females and one male, aged in their twenties and thirties) who had refrained from drinking, eating, or brushing for more than 1 h, by gargling 10 ml saline solution for 30 s.

Half of each oral wash sample was directly subjected to genomic DNA (gDNA) extraction, while the other half was labeled with pHrodoRed and incubated with primary human B cells (4×10⁵ cells per donor, two donors) for 1 h at 37 °C at an MOI of 100. A subset (10⁵ cells) was analyzed by flow cytometry to assess bacterial internalization. The remaining cells were treated with gentamicin (500 μg/ml), lysozyme (300 μg/ml), and DNase I (1 μg) at 37 °C for 1 h to remove extracellular bacterial DNA, followed by DNase I heat inactivation (95 °C, 10 min). gDNA was extracted using the DNeasy PowerSoil Kit (Qiagen, no. 12888-100).

The V3-V4 regions of the bacterial 16S rRNA gene were PCR-amplified, purified, pooled, and sequenced on an Illumina MiSeq. Raw read quality was assessed using VSEARCH based on Phred scores. Paired-end reads were processed in QIIME 2 with DADA2 for amplicon sequence variant (ASV) identification. Taxonomic classification was performed using the Human Oral Microbiome Database (HOMD v15.23). Beta diversity was calculated using Bray-Curtis dissimilarity, visualized by multidimensional scaling, and statistically evaluated by PERMANOVA using the vegan R package.

### 1-9. Bulk RNA-Seq and Data Analysis

Isolated human B cells (2×10⁷) were incubated with pHrodoRed-labeled Pm (MOI 100, 2 h) and sorted into invaded (pHrodo⁺, Pm⁺) and non-invaded (pHrodo⁻, Pm⁻) populations using a SONY SH800S Cell Sorter. Noninfected B cells (NI) served as controls. Murine B cells (10⁶) were incubated with either anti-IgM (10 μg/ml; clone R6-60.2, BD Biosciences, no. 553405) or Ec (MOI 100) for 16 h, with NI controls in parallel. Total RNA was extracted with TRIzol, quality-checked by Bioanalyzer, and quantified by spectrophotometry. Libraries were prepared from 500 ng RNA using the QuantSeq 3′ mRNA-Seq Kit (Lexogen GmbH, no. 191.24), purified, and sequenced (single-end, 75 bp) on an Illumina NextSeq 500.

Reads were pseudo-aligned to the human or mouse transcriptomes using Salmon (v1.10.1). Transcript-to-gene mapping and gene-level quantification were performed with tximport (v1.30.0). A Euclidean distance matrix was generated, and principal coordinate analysis (PCoA) visualized sample relationships. DEGs were identified using DESeq2 (v1.40.2), with adjusted p<0.05 and |log₂ fold change|>1 as significance criteria. GO enrichment analysis was conducted using clusterProfiler (v4.10.1) with gene annotations from org.Mm.eg.db (v3.17.0). Functional enrichment was visualized using enrichplot (v1.22.0) and ggplot2 (v3.5.0).

1-10. scRNA-seq and Data Analysis

Isolated human B cells (1×10⁷; n=3 donors) were incubated with pHrodoRed-labeled Pm at an MOI of 100 for 72 h. Gentamicin (50 μg/ml) was added 1 h post-infection, followed by a secondary infection (MOI 100, 2 h). One sample with <50% viability was excluded, leaving NI B cells from 3 donors and Pm-treated B cells from 2 donors for analysis.

scRNA-seq was performed by GENIUS (Seoul, Korea) using the 10× Genomics Chromium Controller. GEMs were generated, reverse-transcribed, and sequenced on an Illumina platform. Cell Ranger (v7.0.0) aligned reads to GRCh38 and filtered cells (<200 genes, >10% mitochondrial UMIs, >3,000 genes/doublets), retaining 18,285 cells.

Analysis in Seurat (v4.1.0) included normalization, log-transformation, selection of 2,000 variable genes, integration by Canonical Correlation Analysis (CCA), Principal Component Analysis (PCA), UMAP embedding, and clustering (resolution 0.3). Cell types were annotated by reference to published immune datasets and B cell subset markers.

Intracellular Pm transcripts were detected using the SAHMI pipeline. FASTQ reads were classified with Kraken2Uniq (using Prevotella melaninogenica genome GCF_000144405.1), processed with SAHMI's k-mer correlation and background quantile tests, and mapped to 10× barcodes to define Pm⁺ cells.

Differential expression (Pm vs. NI; Pm⁺ vs. NI) was analyzed using Seurat's FindMarkers (log₂FC>0.25; adjusted p<0.05). GO enrichment was performed with clusterProfiler (adjusted p<0.05).

Pseudotime trajectories were constructed using Monocle 3 (v0.2.3.0), setting naïve B cells (clusters 0 and 1) as root cells, with resolution=0.001 in cluster_cells.

### 1-11. Mass Cytometry and Data Analysis

High-dimensional mass cytometry was used to profile human B cells incubated with pHrodo-labeled bacteria. Cells were barcoded to minimize batch variation and stained with 21 lanthanide-conjugated surface marker antibodies (Table S2) following the Maxpar Cell Surface Staining with Fresh Fix protocol (Standard BioTools). After staining, cells were fixed with 1.6% formaldehyde and labeled with Cell-ID Intercalator (Stnadard Biotools no. 201192A). After washing, samples were acquired on a Helios mass cytometer with EQ four-element calibration beads for normalization.

Raw data were debarcoded, arcsinh-transformed, and filtered to remove beads, doublets, and dead cells. Automated clustering was performed with FlowSOM (v4.1.0), generating 10 meta-clusters based on marker expression. Dimensionality reduction and visualization were done using t-SNE. All analyses and plots were generated in FlowJo_v10.10.0 (TreeStar).

### 1-12. In Vitro Functional Assays of Bacteria-Containing B Cells

Human B cells (1×10⁷) were labeled with 5 μM CFSE (Sigma-Aldrich no. 92846) at 37 °C for 10 min in the dark. CFSE-labeled B cells were then incubated with bacteria (MOI 100) for 72 h in RPMI 1640 supplemented with 10% FBS, 50 ng/ml IL-2 (PEPROTECH no. 200-02), and 100 ng/ml BAFF (PEPROTECH no. 310-13) (42). To inhibit bacterial overgrowth and eliminate extracellular bacteria, 50 μg/ml gentamicin was added 1 h after infection. B cells treated with 1 μM CpG ODN 2006 (InvivoGen no. Tlrl-2006-5) served as a positive control. Cell proliferation was assessed by CFSE dilution using flow cytometry (LSRFortessa X-20, BD Biosciences). Culture supernatants were collected to evaluate antibody and chemokine/cytokine production.

### 1-13. Cytokine Array

Cytokines in culture supernatants were detected using the Human Cytokine Array kit (R&D Systems no. ARY005B) according to the manufacturer's protocol. Supernatants from three pooled samples were applied to the array. Mean spot intensity was quantified by densitometry using Bio-Rad Image Lab software and normalized by reference spots.

### 1-14. Enzyme-Linked Immunosorbent Assay (ELISA)

Human IgG and IgM levels in culture supernatants were quantified using the Human IgG (Total) and IgM Uncoated ELISA Kits (Invitrogen no. BMS2091 and no. BMS2098, respectively) according to the manufacturer's instructions. Cytokine levels, including IL-16, IL-18, and TNF-α, were measured using ELISA kits from R&D Systems (no. D1600, DY318-05, and DTA00D, respectively).

Mouse IgG and IgM levels in sera, as well as IFN-7 and IL-17 in culture supernatants, were measured using ELISA Kits from Invitrogen (Mouse IgG: no. 88-50400-88; Mouse IgM: 88-50470-88; Mouse IFN-7: 88-7314-88; Mouse IL-17: 88-7371-88).

For bacteria-specific antibody detection, high-binding 96-well ELISA plates (Corning Costar) were coated overnight at 4 °C with bacterial lysates (2 μg/ml in PBS). Plates were washed with PBS containing 0.05% Tween 20 (PBST) and blocked with 1% BSA in PBS. Mouse serum (1:200 dilutions in blocking buffer) was added in duplicate and incubated for 2 h at room temperature. After washing, wells were incubated with HRP-conjugated anti-mouse IgG or IgM secondary antibodies (Thermo Fisher Scientific) for 1 h. Signals were developed using TMB substrate, stopped with 2 N H₂SO₄, and absorbance was measured at 450 nm using a microplate reader.

### 1-15. Bacteria Survival Assay

Isolated human B cells (2.5×10⁵) were incubated with bacteria at an MOI of 100 for 72 h, with 50 μg/ml gentamicin added 1 h post-infection to kill extracellular bacteria. For CFU enumeration, cells were lysed with 0.5% saponin for 10 min and centrifuged at 10,000×g for 3 min. The resulting bacterial pellets were resuspended in 100 μl PBS and plated onto appropriate agar media according to bacterial species. Intracellular viable bacteria were quantified by colony counting after incubation.

### 1-16. Murine Air Pouch Model

Six-week-old female C57BL/6 (CD45.2) mice were purchased from the Orient Bio, while OVA-specific T cell receptor (TCR) transgenic OT-II [B6.Cg-Tg(TcraTcrb)425Cbn/J] mice (Immunol. Cell Biol., 76, 34-40), on CD45.1 congenic background (CD45.1;OT-II), were kindly provided by Professor Youn-soo Choi (College of Medicine, Seoul National University) and maintained at the Animal Facility of the Seoul National University School of Dentistry. All animal experiments were approved by the Seoul National University Institutional Animal Care and Use Committee (no. SNU-241216-1-1, SNU-24507-3-2, and SNU-230908-4-1). Only female mice aged 7-8 weeks were used.

Animals were randomly assigned to experimental groups at the beginning of each experiment. Investigators were not blinded to group allocation, and sample size was not predetermined due to the absence of prior data. All results were validated in three independent experiments using 2-4 mice per group, and pooled data from three experiments (n=8-10 mice per group) are presented.

A sterile air pouch was created in 7-week-old C57BL/6 (CD45.2⁺) mice by subcutaneously injection of 5 ml sterile air into the dorsal region using a 26G needle on day −7, followed by a booster injection of 3 ml air on day −2. For B-cell transfer experiments conducted on day 0, splenic B cells from CD45.1⁺ mice were isolated and stained with 2 μM CFSE for 10 min at 37 °C The CFSE-labeled cells were then incubated with pHrodoRed-labeled bacteria at an MOI of 100 for 1 h at 37 °C, washed with PBS, and 1×10⁶ cells were transferred into the dorsal air pouch of each recipient mouse.

Mice were euthanized 2 h, 1, 5, or 14 days post-injection (dpi). To assess immune cell recruitment, air pouch lavage fluid was collected by injecting 3 ml of 50 mM EDTA-PBS, followed by gentle massage for 30 s. The lavage fluid was collected and centrifuged at 400×g for 5 min.

To assess B- and T-cell activation, cervical, axillary, brachial, and inguinal LNs (draining lymph node; D.LNs), mesenteric LNs (non-draining lymph node; ND.LNs), and spleen were collected for single-cell preparation. To evaluate bacterial survival within transferred B cells in vivo, cells from lavage fluid (10% of total), lymph nodes (1×10⁶), and spleen (1×10⁶) were lysed in 0.5% saponin for 10 min at room temperature. Lysates were plated on appropriate agar plates and incubated for 1-3 days under aerobic conditions at 37 °C Colony counts were used to determine the number of viable intracellular bacteria.

### 1-17. Flow Cytometry

Bacterial internalization by B cells was assessed based on pHrodoRed fluorescence, excluding dead cells labeled with Ghost Dye Violet 510 (TONBO Biosciences no. 13-0870-T100). For surface marker analysis, infected human B cells were stained with Ghost Dye Violet 510 for 15 min at 4 °C, washed with FACS buffer (PBS+2% FBS), blocked with Fc receptor antibody (BioLegend no. 422302), and with BV 650 anti-human CD27 (clone 323, BioLegend no. 302827) and APC/Cy7 anti-human IgD (clone IA6-2, BioLegend no. 348217).

To assess Syk activation, infected human or mouse B cells were stained with APC anti-anti-human/mouse phospho-Syk (clone moch1ct, Invitrogen no. 17-9014-42).

Mouse cells were stained with multiparameter antibodies after Ghost Dye Violet 510 labeling and Fc blocking (anti-mouse CD16/32; clone 2.4G2, BD Bioscience no. 553142). For leukocyte recruitment analysis, lavage cells were stained with AF 647 Annexin V (BioLegend no. 640953), PerCP anti-mouse CD19 (clones 6D5, BioLegend no. 115532), Pacific Blue anti-mouse CD3F (clone KT3.1.1, BioLegend no. 155611), PE/Cy7 anti-mouse CD11b (clone M1/70, BioLegend no. 101215), BV605 anti-mouse Ly-6G (clone 1A8, BioLegend no. 127639), and APC anti-mouse CD45.1 (clone A20, BioLegend no. 110714). To analyze B-cell activation, cells (1×10⁶) were stained with PerCP anti-mouse CD19 (clone 6D5), PE/Cy7 anti-mouse CD80 (clone 16-10A1, BioLegend no. 104733), PE/Cy7 anti-mouse CD86 (clone GL-1, eBioscience no. 560582), BV605 anti-mouse I-A/I-E (clone M5/114.15.2, BioLegend no. 107639), APC anti-mouse CD45.1 (clone A20, BioLegend no. 110714), Pacific Blue CD3F (clone KT3.1.1, BioLegend no. 155611), and FITC anti-mouse CD4 (clone H129.19, BioLegend no. 100405).

For T-cell activation analysis, cells (1×10⁶) were stimulated with 50 ng/ml phorbol 12-myristate 13-acetate, 1 g/ml ionomycin, and Brefeldin A (Sigma-Aldrich no. 20350-15-6) for 4 h. Following stimulation, surface staining was performed using Pacific Blue anti-mouse CD3F (clone KT3.1.1, BioLegend no. 155611), BV650 anti-mouse/human CD44 (clone IM7, BioLegend no. 103049), APC-eFluor 780 anti-mouse CD4 (clone GK1.5, Invitrogen no. 47-0041082), and PE anti-mouse CD19 (clone 6D5, BioLegend no. 115508). Cells were then fixed and permeabilized using the eBioscience™ Foxp3/Transcription Factor Staining Buffer Set (Invitrogen no. 00-5523-00) and stained with FITC anti-mouse IFNγ (clone XMG1.2, BioLegend no. 505805), PE/Cy7 anti-mouse IL-17 (clone TC11-18H10.1, BioLegend no. 506921), and AF647 anti-mouse Foxp3 (clone MF-14, BioLegend no. 126408) for 45 min at room temperature. Cells were washed with permeabilization buffer and resuspended in FACs buffer.

Flow cytometry acquisition was performed using an LSRFortessa X-20 (BD Biosciences), and data were analyzed with FlowJo_v10.8.1.

### 1-18. In Vitro Assays of Antigen Presentation to T Cells

Isolated mouse B cells (1×10⁶) were infected with Pm, Ec, Se, OTII-Pg, or pTCOW-Pg at an MOI of 100 for 1 h at 37 °C Gentamicin (50 μg/ml) was then added to eliminate extracellular bacteria. The infected B cells were co-cultured with wild-type or OT-II CD4⁺ T cells (1×10⁶), isolated using the CD4 T-cell isolation kit (STEMCELL Technologies, no. 19852), in RPMI 1640 for 4 days, with brefeldin A added during the final 4 h. B cells incubated with OT-II peptide (2 μg/ml) and LPS (2 μg/ml) served as a positive control for OT-II T-cell activation.

Conventional DCs were differentiated by culturing whole bone marrow cells in RPMI 1640 supplemented with 10% FBS, 1% penicillin/streptomycin, 55 M β-mercaptoethanol, and 5% Flt3L conditioned medium for 9 days. DCs (2×10⁵) were infected with bacteria at an MOI of 100 for 1 h and subsequently co-cultured with CD4⁺ T cells under the same conditions.

After co-culture, cells were harvested for flow cytometric analysis, and supernatants were collected for IL-17 and IFN-γ ELISA.

### 1-19. Statistical Analysis

Data analysis and visualization were performed using GraphPad Prism (v8.0.2 and v9.3.0; GraphPad Software) or R (v4.5.0). Most data from animal experiments satisfied normal distribution, and in vitro data were assumed to follow a normal distribution. For comparisons between two independent groups (NI vs. bacteria-containing B cells), a two-tailed Student's t-test was used. For comparisons among three or more groups, one-way ANOVA with Bonferroni correction was applied. No data were excluded. A p-value<0.05 was considered statistically significant, while 0.05<p<0.1 indicated a trend. Detailed statistical information for each experiment is provided in the figure legends.

### Example 2

### 2. Human B Cells Internalize Bacteria in Tissue and In Vitro

To explore interactions between B cells and bacteria in tissues, sections from periodontitis lesions and labial salivary glands of SjD patients were analyzed using CD20 immunohistochemistry and in situ hybridization for 16S rRNA. Bacteria were frequently detected within infiltrated CD20⁺ B cells (5/5 periodontitis lesions and 5/6 SjD salivary gland cases), suggesting potential bacterial internalization (A of FIG. 1).

To assess whether B cells can internalize bacteria, several bacterial species associated with periodontitis or SjD (Table 2) were labeled with pHrodoRed, a pH-sensitive dye, and incubated with primary human B cells at a multiplicity of infection (MOI) of 10 or 100 for 1 or 16 h (B of FIG. 1).

TABLE 2

| Bacterial species | Association with diseases |
|---|---|
| Staphylococcus aureus | Most abundant within gingival tissue |
| ATCC 29213 (Sa) | cells, newly identified periodontitis |
| | pathogen |
| Staphylococcus | Quite frequently identified in the salivary |
| epidermidis KCTC 1917 | gland proteomics dataset |
| (Se) | |
| Streptococcus oralis | Oral commensal, most frequently |
| ATCC 9811 (So) | identified in the salivary gland |
| | proteomics dataset |
| Streptococcus salivarius | Oral commensal |
| KCTC 5512 (Ss) | |
| Rothia mucilaginosa | Oral species associated with Sjogren's |
| KCTC 19862 (Rm) | disease |
| Escherichia coli 7.2 (Ec) | Isolated from oral lichen planus biopsy, |
| | newly identified periodontitis pathogen |
| Porphylomonas gingivalis | Periodontitis pathogen |
| ATCC 33277 (Pg) | |
| Prevotella melaninogenica | Oral species associated with Sjogren's |
| KCTC 5457 (Pm) | disease |
| Veillonella dispar | Oral species associated with Sjogren's |
| ATCC 17748 (Vd) | disease |

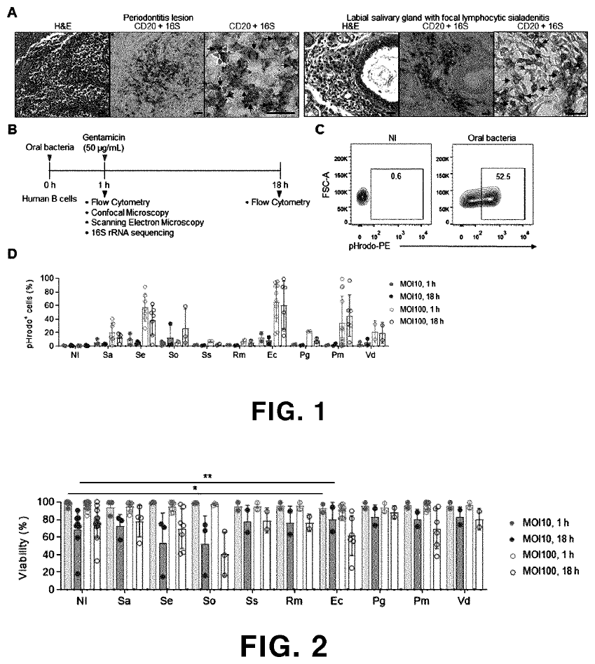

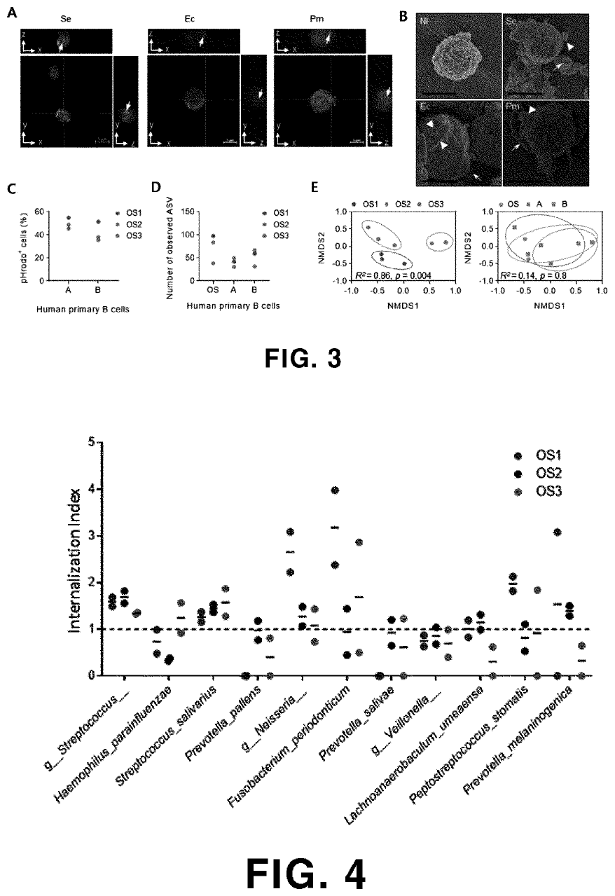

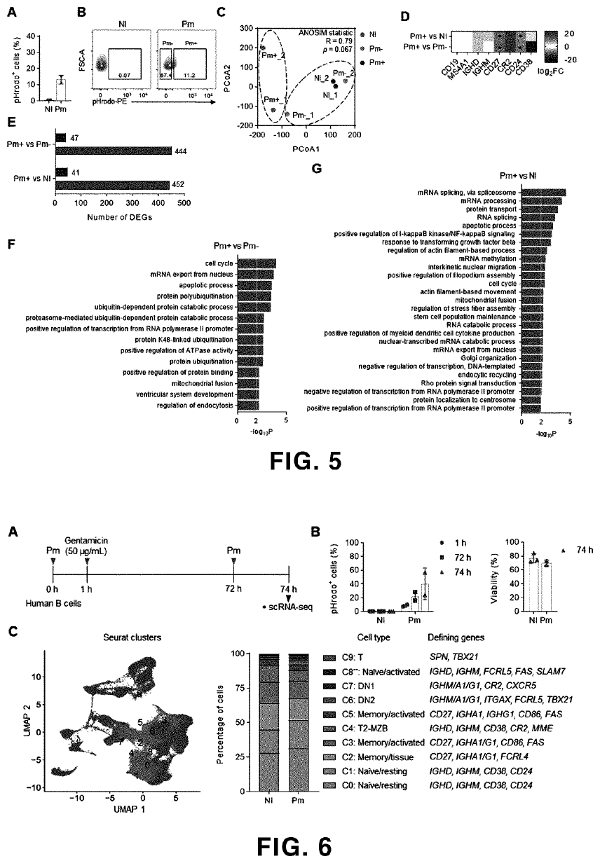

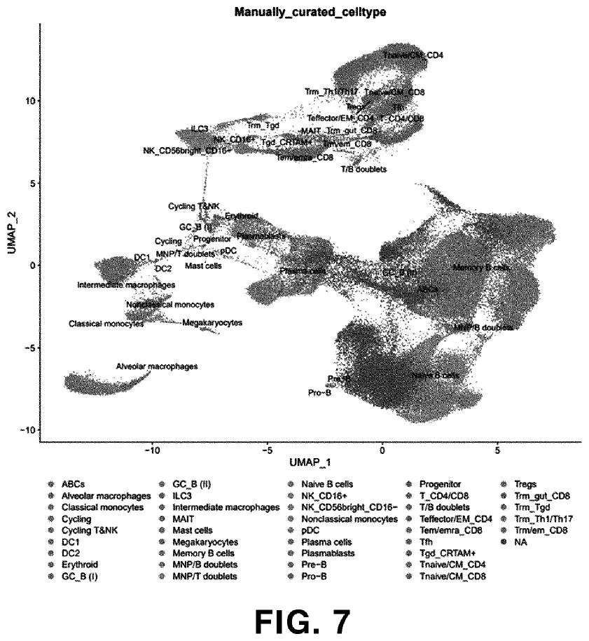

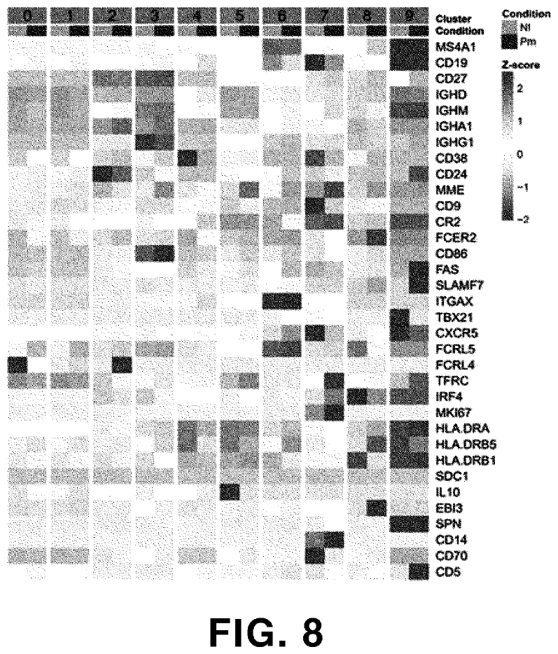

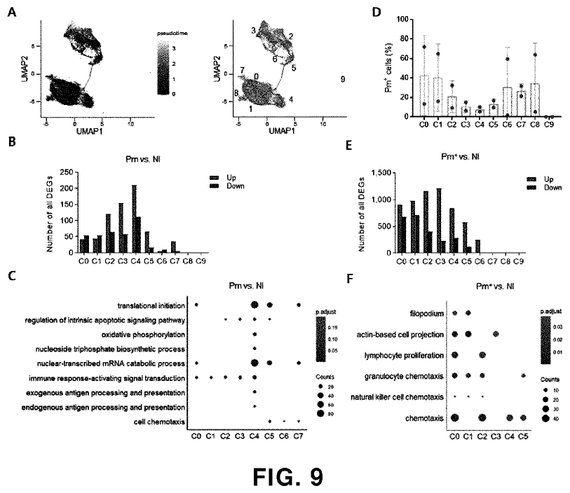

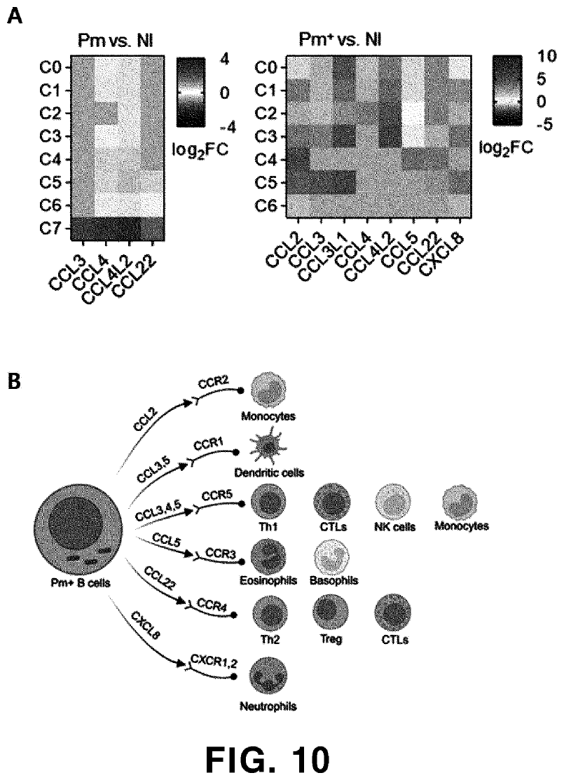

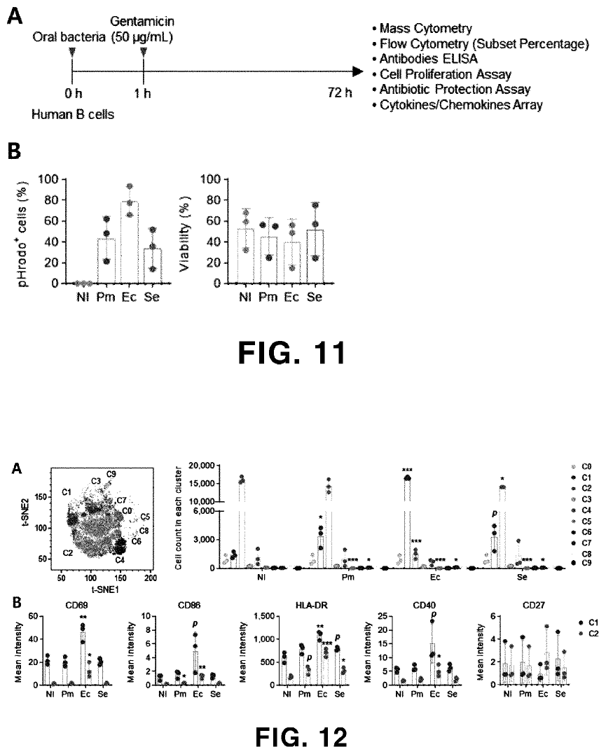

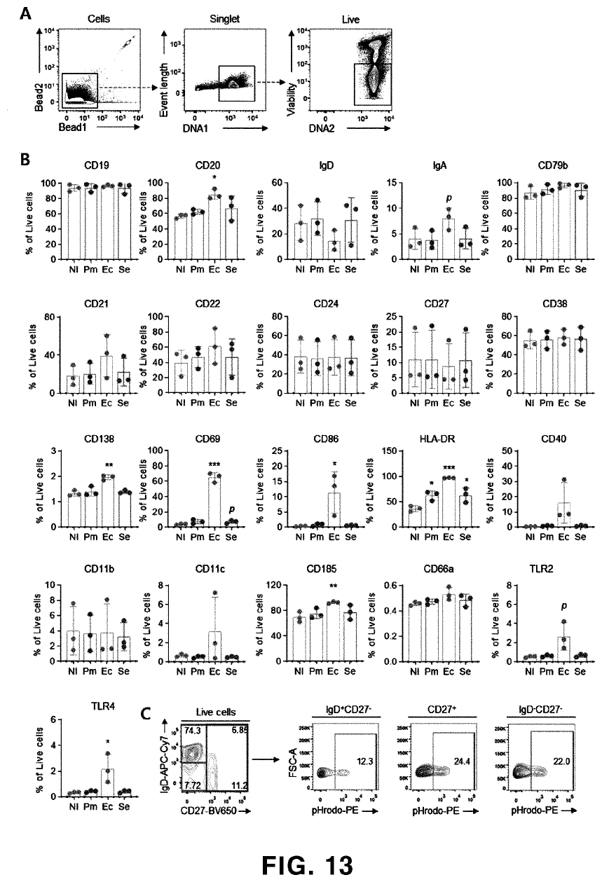

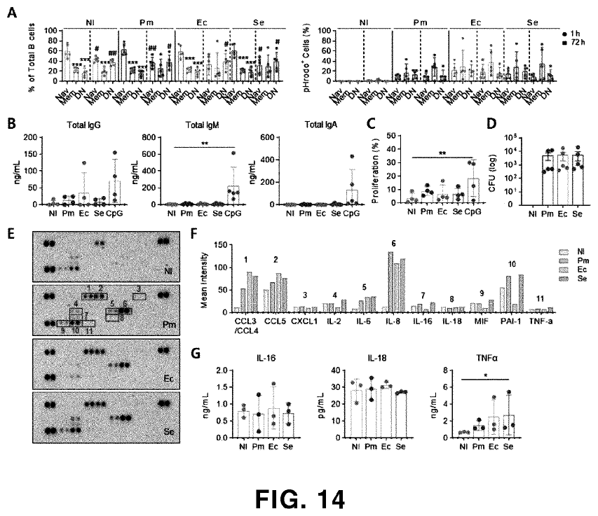

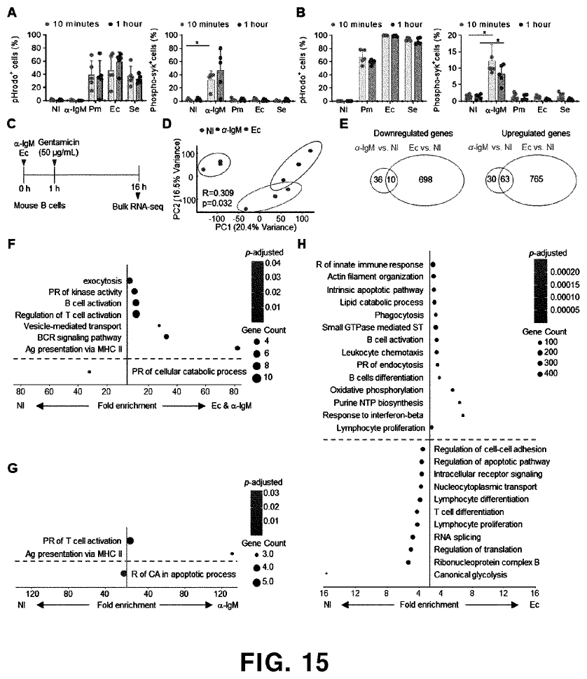

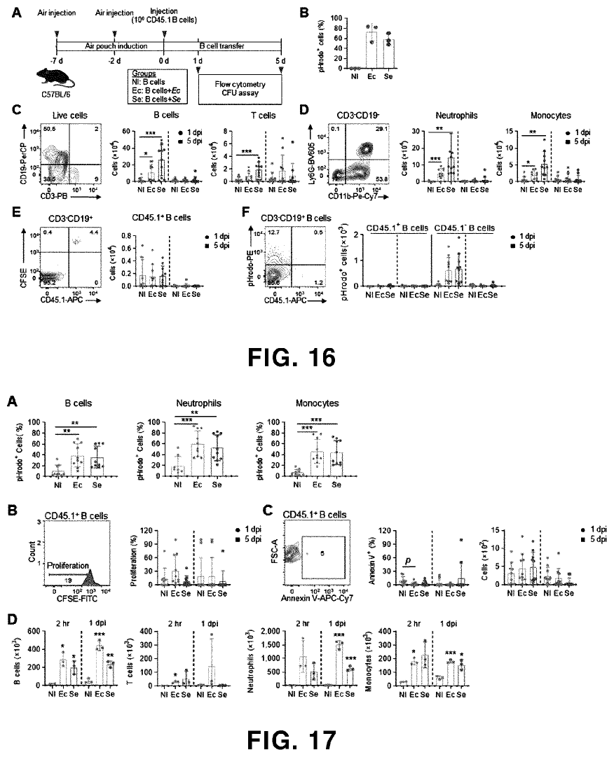

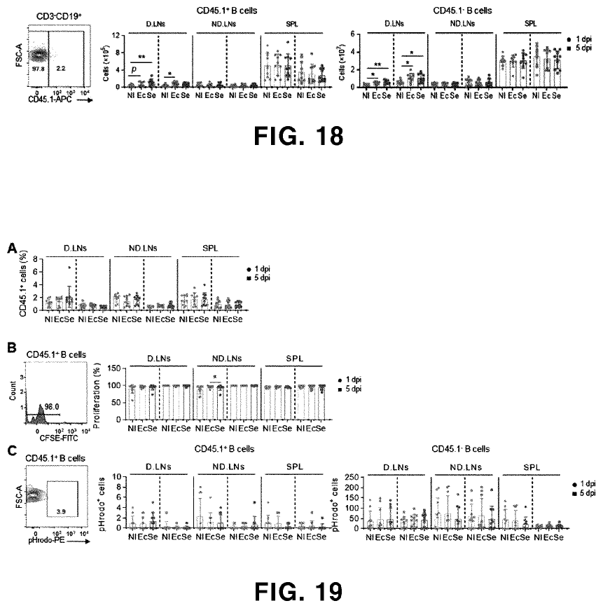

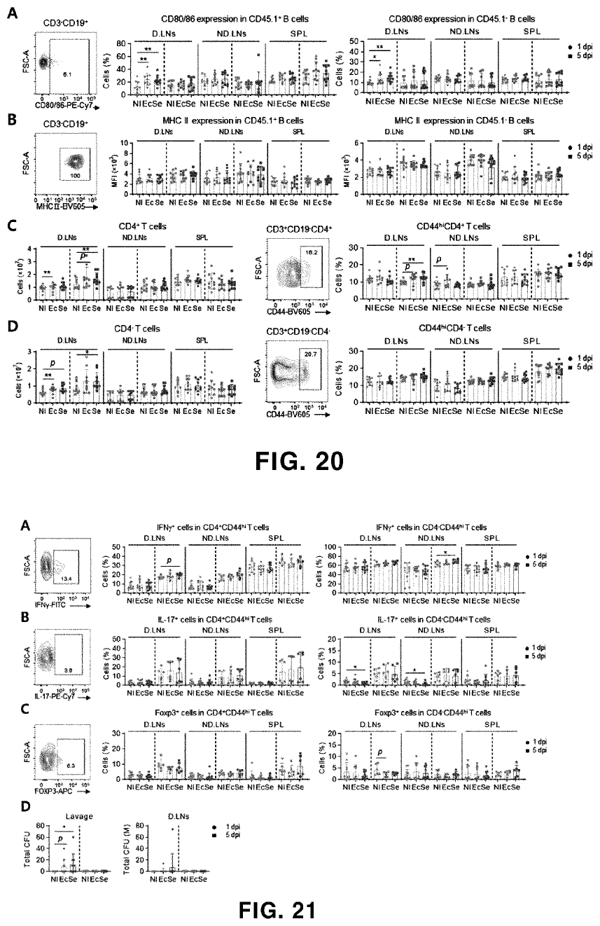

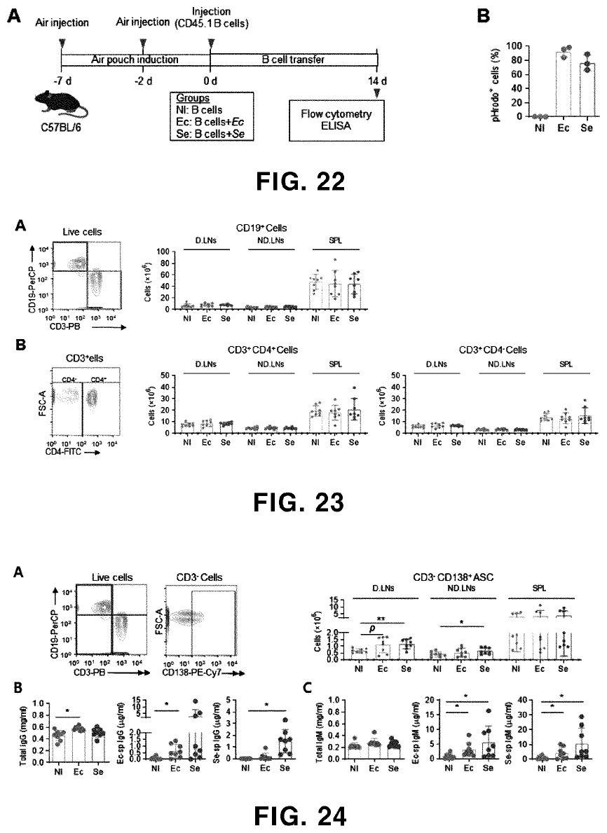

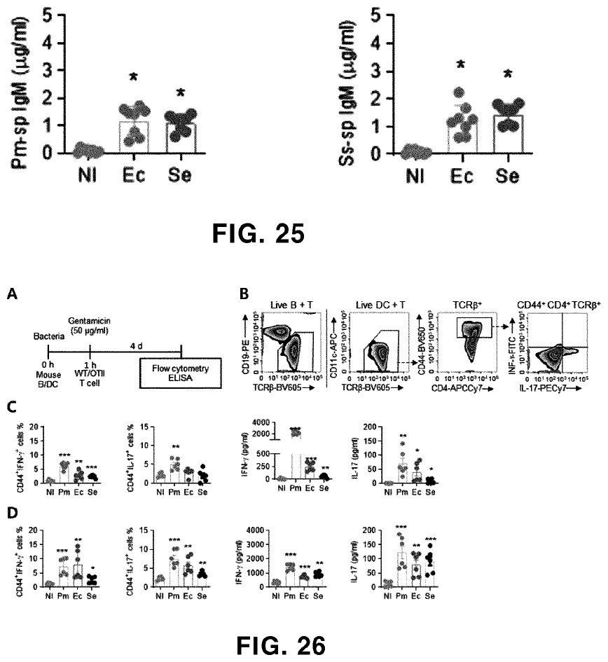

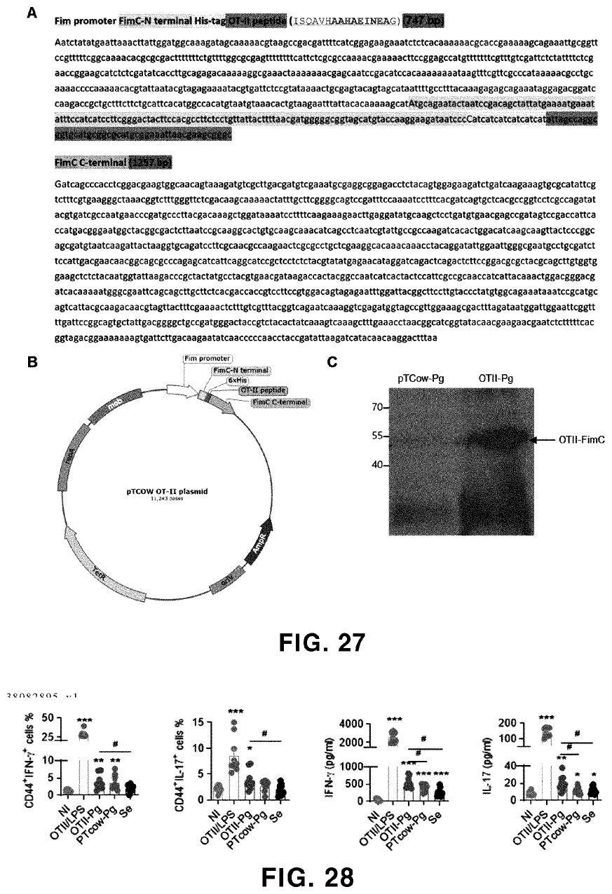

Flow cytometry revealed variable bacterial uptake (0.3% to 99.3% pHrodo⁺ cells), depending on the species, donor, and MOI (C and D of FIG. 1). A 1 h incubation was generally sufficient for maximal internalization with no significant impact on viability, except in response to Escherichia coli (FIG. 2). Internalization of E. coli (Ec), Staphylococcus epidermidis (Se), and Prevotella melaninogenica (Pm) was further confirmed by confocal and scanning electron microscopy (A and B of FIG. 3).

To assess the uptake of complex microbiota, three oral wash samples were incubated with B cells from two donors. pHrodo labeling indicated that 36-55% of B cells internalized bacteria (C of FIG. 3). 16S rRNA gene sequencing revealed that 42-82% of bacterial species present in oral washes were successfully internalized (D of FIG. 3). Bray-Curtis dissimilarity analysis showed clustering by oral source, but no clear separation between the original oral wash and the intra-B cell microbiota (E of FIG. 3). Internalization indexes calculated for 11 species consistently detected across all three oral samples varied by B cell donor and microbial source (FIG. 4).

Collectively, human B cells internalize diverse bacterial species, both individually and from complex communities.

### Example 3

### 3. Bacterial Internalization Induces Distinct Transcriptional Programs in B Cells

To investigate the transcriptional features of B cells capable of internalizing bacteria, we incubated B cells with pHrodoRed-labeled Pm for 2 h and sorted into Pm⁻ (exposed but non-internalizing) and Pm⁺ (bacteria-containing) populations. Untreated cells served as non-infected (NI) controls (A and B of FIG. 5). Bulk RNA sequencing (RNA-seq) identified a total of 8,139 differentially expressed genes (DEGs; p<0.05) across three comparisons (Pm⁺ vs. NI, Pm⁺ vs. Pm⁻, and Pm⁻ vs. NI). Principal coordinate analysis (PCoA) showed a trend toward the distinct separation of Pm⁺ cells (ANOSIM R=0.79, p=0.067; C of FIG. 5). This suggests the transcriptional specificity of the internalized cells. Interestingly, the expression of B-cell subset-defining markers, such as CD27 and CD24, was exclusive to Pm⁺ cells. While this could suggest selective internalization by memory B cells, the absence of CD27 and IGHD in NI samples supports the interpretation of a stimulus-induced phenotypic shift (D of FIG. 5). Gene Ontology (GO) analysis of DEGs (q<0.01; genes expressed in >50% of samples) indicated that Pm⁺ cells were enriched for pathways related to actin filament-based processes, filopodium assembly, endocytosis, mitochondrial fusion, apoptosis, the cell cycle, and mRNA processing, consistent with the observed morphological changes (E to G of FIG. 5).

To assess longer-term, subset-specific transcriptional changes, B cells were infected with Pm for 72 h, followed by a 2 h re-infection, and analyzed by single-cell RNA-seq (scRNA-seq) (A and B of FIG. 6). The re-infection step was included to recapitulate the acute internalization signatures identified in the bulk RNA-seq data. Seurat clustering identified 10 cell populations (C0-C8 as B cells, C9 as contaminating T cells; C of FIG. 6 and FIG. 7), with cluster identities assigned based on canonical subset markers. Among these, only the activated naïve B cell cluster (C8) expanded significantly upon Pm infection (C of FIG. 6). Trajectory analysis revealed differentiation branches leading from resting naïve toward activated naïve/double negative 1 (DN1) B cells, and from tissue-like memory toward activated memory/DN2 B cells (A of FIG. 9). Differentiation trajectories were similar between NI and Pm-infected conditions, indicating that Pm-internalization does not fundamentally alter established B-cell lineages.

A comparison between Pm-infected and NI cells revealed the largest transcriptional shifts in clusters C2-C4; specifically, C4 showed significant enrichment for multiple GO terms, including antigen presentation and oxidative phosphorylation (B and C of FIG. 9). Using the single-cell analysis of host-microbiome interactions (SAHMI) pipeline to detect microbial transcripts within host data, we identified Pm⁺ cells across all clusters (D of FIG. 9). Given that intracellular Pm losing viability during culture would likely cease transcript production, the re-infection step likely enhanced the detection of recently internalized bacteria. DEGs in the Pm⁺ vs. NI comparison were more evenly distributed across C0-C4 and were enriched for pathways involved in leukocyte chemotaxis, lymphocyte proliferation, actin-based cell projection, and filopodium assembly (FIG. 2, H and I), reinforcing the findings from our bulk RNA-seq analysis.

Chemokines such as CCL3, CCL4, CCL5, CCL22, and CXCL-8/IL-8 were upregulated in Pm and Pm⁺ groups and broadly expressed across clusters (A and B of FIG. 10), suggesting potential to recruit monocyte, neutrophils, DCs, Th1 cells, Th2/Treg cells, cytotoxic T lymphocytes (CTL), and NK cells. Consistent with the bulk RNA-seq findings, CD27 was differentially expressed in Pm⁺ cells from the resting naïve B cell populations C0 (2.2-fold) and C1 (1.4-fold) (adjusted p<0.0001). This supports that bacterial internalization induces activation and phenotypic changes in B cells.

Collectively, these data indicate that bacterial internalization induces broad, subset-specific transcriptional reprogramming in B cells, characterized by endocytic activity, enhanced antigen presentation, and chemokine production.

### Example 4

### 4. Bacteria-Containing B Cells Exhibit an APC-Like Phenotype and Secrete Chemokines

To assess protein-level changes, B cells were incubated with pHrodoRed-labeled Pm, Ec, or Se for 72 h and analyzed by mass cytometry using a panel of 21 antibodies (A and B of FIG. 11). FlowSOM clustering identified 10 phenotypic clusters. Among these, C1 represented an activated B cell population characterized by high expression of CD69, CD86, HLA-DR, and CD40; the frequency of this cluster increased in Pm- and Ec-infected samples. In contrast, C2, a less activated cluster, was reduced by Ec and Se infection (A and B of FIG. 12). Furthermore, bacterial infection led to the upregulation of CD86, HLA-DR, and CD40 within both C1 and C2, indicating enhanced potential for antigen presentation (B of FIG. 12).

However, comparable levels of CD27 expression between C1 and C2 suggest that both clusters contain a similar proportion of memory B cells. Additionally, infection with Ec, the species showing the highest internalization efficiency, significantly increased the CD20⁺, CD138⁺, CD185⁺, and TLR4⁺ populations, consistent with B-cell activation and differentiation into plasma cells (A and B of FIG. 13).

Guided by the RNA-seq findings, we used flow cytometry to determine whether bacteria are preferentially internalized by naïve B cells and whether this induces a transition to CD27⁺ memory phenotype (C of FIG. 13). The naïve B-cell subset, which was predominant at 1 hour, declined over 72 h of culture, accompanied by a corresponding increase in DN subsets. Notably, while the memory B-cell subset remained significantly smaller than the naïve subset in NI cells after 72 h, this difference was abolished in bacteria-infected samples (A of FIG. 14). Although the specific B-cell subset that preferentially internalized bacteria varied among donors, the percentage of pHrodo-positive cells trended higher only within the memory B-cell subset after 72 h (A of FIG. 14).

Despite the known capacity of TLR9 engagement to induce antibody secretion and proliferation in B cells, bacterial infection did not elicit these responses to a significant extent compared to NI controls (B and C of FIG. 14). Antibiotic protection assays revealed that internalized bacteria were not completely cleared even after 72 h (D of FIG. 14). To validate the chemokine-related transcriptional responses, cytokine array analysis of culture supernatants was performed, revealing robust secretion of CCL3/CCL4, CCL5, IL-8, and IL-6 in response to all three bacterial species. PAI-1 was upregulated by Pm and Se but downregulated by Ec (E and F of FIG. 14). Although IL-16, IL-18, and TNF-α were only weakly detected by the array, follow-up ELISA confirmed elevated TNF-α production exclusively in Se-infected samples (G of FIG. 14).

Collectively, these data indicate that bacteria-containing B cells exhibit an activated APC-like phenotype, secrete pro-inflammatory chemokines but possess a limited capacity to eliminate intracellular bacteria.

### Example 5

### 5. Bacterial Internalization Induces Transcriptional Programs Distinct from BCR Activation

Given the high efficiency of internalization observed, the present inventors hypothesized that the process occurs through a BCR-independent mechanism. Consistent with this, phosphorylation of Syk, a hallmark of BCR signaling, was induced by anti-IgM stimulation but not by bacterial infection in either human or mouse B cells (A and B of FIG. 15).

To compare transcriptional responses, mouse B cells were left untreated, stimulated with anti-IgM, or infected with Ec for 16 h prior to bulk RNA-seq (C of FIG. 15). PCoA revealed three distinct transcriptomic profiles (D of FIG. 15). Infection with Ec induced substantially more DEGs than anti-IgM stimulation, likely reflecting the higher proportion of affected cells (over 90% vs. <15%), with only modest overlap between the two groups (E of FIG. 15). Shared GO terms included upregulation of antigen presentation via MHC II, BCR signaling, vesicle trafficking, T cell activation, and B cell activation (F of FIG. 15). Anti-IgM-specific upregulated DEGs were further enriched for antigen presentation and T cell activation, whereas genes involved in apoptotic regulation were downregulated (G of FIG. 15). In contrast, Ec-specific upregulated DEGs were enriched for pathways related to oxidative phosphorylation, leukocyte chemotaxis, endocytosis, phagocytosis, actin filament organization, and innate immune activation, closely mirroring the responses observed in Pm-infected human B cells (H of FIG. 15). Notably, the enhanced oxidative phosphorylation and reduced glycolysis observed in Ec-infected cells sharply contrasted with the glycolytic shift typically induced by BCR engagement, highlighting a divergent metabolic profile.

Together, these findings demonstrate that bacterial internalization triggers a broad, BCR-independent transcriptional program characterized by metabolic reprogramming and enhanced endo-phagocytic activity.

### Example 6

### 6. Bacteria-Containing B Cells Activate T Cells In Vivo and In Vitro

To evaluate the in vivo function of B cells harboring internalized bacteria, we injected carboxyfluorescein succinimidyl ester (CFSE)-labeled CD45.1⁺ B cells containing either Ec or Se into the air pouches of C57BL/6 mice; NI B cells served as controls. Pm was excluded from in vivo studies due to biosafety level 2 restrictions. We subsequently assessed leukocyte recruitment in air pouch lavage fluid and the activation of B and T cells in the lymph nodes (LNs) and spleen at 1 and 5 days post-injection (dpi) (A and B of FIG. 16).

Within the air pouch, although only a small fraction of the donor B cells remained at the site by 1 dpi, Ec- and Se-containing B cells induced significantly greater recruitment of host leukocytes, including B cells, T cells, neutrophils, and monocytes, compared to NI controls. By 5 dpi, most recruited leukocytes had cleared, with the exception of T cells (C to E of FIG. 16). Unexpectedly, at 1 dpi, pHrodoRed-labeled bacteria were predominantly detected within the endogenous CD45.1⁻ B cell population, at levels comparable to those observed in neutrophils and monocytes, suggesting bacterial transmission from donor (CD45.1⁺) B cells to recruited host cells (F of FIG. 16 and A of FIG. 17). Notably, bacterial internalization did not significantly affect the proliferation or apoptosis of the donor CD45.1⁺ B cells (B and C of FIG. 17). In an additional experiment, however, leukocyte recruitment by bacteria-containing B cells was already evident as early as 2 h post-injection (D of FIG. 17).

Donor CD45.1⁺ B cells were detected in both draining and non-draining LNs, as well as the spleen, constituting over 1% of total B cells at 1 dpi; this frequency decreased to less than 1% by 5 dpi (FIG. 18 and A of FIG. 19). Bacterial internalization significantly enhanced donor cell accumulation specifically within draining LNs and led to the upregulation of CD80/CD86 at 1 dpi, while MHC II expression remained high across all B cell groups. By this stage, the majority of CD45.1⁺ B cells in lymphoid tissues had lost both their CFSE-label and pHrodoRed-labeled bacteria (B and C of FIG. 19). Importantly, the injection of bacteria-containing B cells led to a marked increase in both the number and activation of endogenous (CD45.1⁻) B cells in the draining LNs (FIG. 18 and A and B of FIG. 20).

T cell activation was primarily localized to the draining LNs, characterized by an expansion of both CD4⁺ and CD4⁻ T cells and elevated CD44 hi populations by 5 dpi (C and D of FIG. 20). Specifically, injection of Se-containing B cells increased the frequency of IFN-7⁺ cells among CD44^{hi}CD4⁺ and CD44^{hi}CD4⁻ T cells at 5 dpi, whereas IL-17⁺CD44^{hi}CD4⁻ T cells were reduced at 1 dpi (A to C of FIG. 21).

Viable intracellular bacteria were detected only transiently in air pouch lavage and draining LNs of a few mice and were absent from non-draining LNs or the spleen (D of FIG. 21).

We also examined the fate of donor B cells and the resulting antibody response at 14 dpi (FIG. 22). CD45.1⁺ B cells comprised less than 0.1% of total B cells in the LNs and spleen of most mice, with no evidence that bacterial internalization prolonged their survival. The expanded B and T cell populations observed at earlier time points in the draining LNs had contracted by 14 dpi (FIG. 23), while CD138⁺ antibody-secreting cells increased (A of FIG. 24). Ec-containing B cells induced an increase in both total IgG and Ec-specific IgG, whereas Se-containing B cells selectively increased Se-specific IgG (B of FIG. 24). The IgM response was less specific; B cells containing either Ec or Se induced elevated IgM levels against both species as well as unrelated bacteria, such as Pm and Streptococcus salivarius, without significant changes in total IgM (C of FIG. 24 and FIG. 25).

Given that DCs could also take up bacteria during the transmission from donor to host B cells, we compared the T cell activation capacities of B cells and DCs in vitro using flow cytometry and ELISA (A and B of FIG. 26). Bacteria-containing B cells clearly induced IFN-γ or IL-17 production from T cells, whereas NI B cells did not. Pm stimulated the strongest T cell activation through both B cells and DCs, suggesting that a B cell's capacity to activate T cells does not necessarily correlate with its internalization efficiency (C and D of FIG. 26). The ability of B cells to present specific bacterial antigens was further confirmed using OT-II T cells and Porphyromonas gingivalis expressing the OT-II peptide within its FimC protein (OTII-Pg, FIG. 27). The sequence information shown in A of FIG. 27 is as shown in Table 3 below.

TABLE 3

| | | SEQ |
|---|---|---|
| | | ID |
| Sequence name | Nucleotide sequence(5′→3′) | NO. |
| Fim promoter | aatctatatgaattaaacttattggatggcaaagatagcaaaaa | 1 |
| FimC-N | cgtaagccgacgattttcatcggagaagaaatctctcacaaaa | |
| terminal His- | aacgcaccgaaaaagcagaaattgcggttccgtttttcggcaa | |
| tag OT-II | aacacgcgcgactttttttctgttttggcgcgagttttttttcattct | |
| peptide | cgcgccaaaacgaaaaacttccggagccatgtttttttcgtttgt | |
| | cgattctctattttctcgaaccggaagcatctctcgatatcacctt | |
| | gcagagacaaaaaggcgaaactaaaaaaacgagcaatccga | |
| | catccacaaaaaaaataagtttcgttcgcccataaaaacgcctg | |
| | caaaaccccaaaaacacgtattaatacgtagagaaaatacgtg | |
| | attctccgtataaaactgcgagtacagtagcataattttgccttta | |
| | caaagagagcagaaataggagacggatccaagaccgctgctt | |
| | tcttctgcattcacatggccacatgtaatgtaaacactgtaagaa | |
| | tttattacacaaaaagcatatgcagaatactaatccgacagctat | |
| | tatgaaaatgaaatatttccatcatccttcgggactacttccacg | |
| | ccttctcctgttattacttttaacgatgggggcggtagcatgtac | |
| | caaggaagataatccccatcatcatcatcatcatattagccagg | |
| | cggtgcatgcggcgcatgcggaaattaacgaagcgggc | |
| FimC C- | gatcagcccacctcggacgaagtggcaacagtaaagatgtcg | 2 |
| terminal | cttgacgatgtcgaaatgcgaggcggagacctctacagtgga | |
| | gaagatctgatcaagaaagtgcgcatattcgtctttcgtgaagg | |
| | gctaaacggtctttgggttctcgacaagcaaaaactatttgcttc | |
| | ggggcagtccgatttccaaaatcctttcacgatcagtgctcacg | |
| | ccggtcctcgccagatatacgtgatcgccaatgaacccgatgc | |
| | ccttacgacaaagctggataaaatccttttcaagaaagaacttg | |
| | aggatatgcaagctcctgatgtgaacgagccgatagtccgacc | |
| | attcaccatgacgggaatggctacggcgactcttaatccgcaa | |
| | ggcactgtgcaagcaaacatcagcctcaatcgtattgccgcca | |
| | agatcacactggacatcaagcaagttactcccggcagcgatgt | |
| | aatcaagattactaaggtgcagatccttcgcaacgccaagaac | |
| | tcgcgcctgctcgaaggcacaaacaaacctacaggatattgga | |
| | attgggcgaatgcctgcgatcttccattgacgaacaacggcag | |
| | cgcccagagcatcattcaggcatccgcgcctctctacgtatatg | |
| | agaacataggatcagactcagactcttccggacgcgctacgca | |
| | gcttgtggtggaagctctctacaatggtattaagacccgctact | |
| | atgcctacgtgaacgataagaccactacggccaatcatcacta | |
| | ctccattcgccgcaaccatcattacaaactggacgggacgatc | |
| | acaaaaatgggcgaattcagcagcttgcttctcacgaccaccg | |
| | tccttccgtggacagtagagaatttggattacggcttccttgtac | |
| | cctatgtggcagaaataaatccgcatgcagtcattacgcaaga | |
| | caacgtagttactttcgaaaactctttgtcgtttacggtcagaatc | |
| | aaaggtcgagatggtagccgttggaaagcgactttagataatg | |
| | gattggaattcggttttgattccggcagtgctattgacggggct | |
| | gccgatgggactaccgtctacactatcaaagtcaaagctttgaa | |
| | acctaacggcatcggtatacaacgaagaacgaatctctttttca | |
| | cggtagacggaaaaaaagtgattcttgacaagaatatcaaccc | |
| | ccaacctaccgatattaagatcatacaacaaggactttaa | |

B cells harboring OTII-Pg induced significant IFN-γ and IL-17 production from OT-II T cells, significantly exceeding the levels induced by control pTCOW-Pg (empty vector) or Se (FIG. 28).

Collectively, these results demonstrate that bacteria-containing B cells can activate local innate and adaptive immune responses and migrate to secondary lymphoid tissues to initiate systemic immunity.

What is claimed is:

1. A method for preparing B cells internalizing bacteria, comprising co-culturing bacteria and isolated B cells.

2. The method of claim 1, wherein the B cells are isolated from human blood.

3. The method of claim 1, wherein the bacteria and the B cells are co-cultured at a multiplicity of infection (MOI) of 5 to 500.

4. The method of claim 1, wherein the concentration of the B cells is 1×10⁵/mL to 1×10⁶/mL.

5. The method of claim 1, wherein the culture is performed for 0.5 to 100 hours.

6. The method of claim 1, wherein the bacteria are internalized into the B cells independently of a B cell receptor (BCR).

7. The method of claim 1, wherein the B cells internalizing the bacteria activate an immune response.

8. The method of claim 1, wherein the B cells internalizing the bacteria have increased phagocytic activity, antigen-presenting ability or chemokine secretion.

9. B cells internalizing bacteria, prepared by the method of claim 1.

10. B cells internalizing bacteria, wherein the B cells have internalized the bacteria independent of a BCR.

11. A method for preventing or treating cancer or bacterial infection, comprising administering the B cells of claim 9 to a subject in need thereof.

12. The method according to claim 11, wherein the cancer is selected from the group consisting of breast cancer, ovarian cancer, uterine cancer, pancreatic cancer, lung cancer, gastric cancer, liver cancer, colorectal cancer, skin cancer, head or neck cancer, brain cancer, laryngeal cancer, prostate cancer, bladder cancer, esophageal cancer, thyroid cancer, kidney cancer, rectal cancer, leukemia and hematological malignancies.

13. A method for inducing an immune response, comprising administering the B cells of claim 9 to a subject in need thereof.


SEQUENCE LISTING (2 sequences)

SEQ ID NO: 1 - DNA, 747 bp, synthetic construct
  source 1..747: mol_type = other DNA
aatctatatgaattaaacttattggatggcaaagatagcaaaaacgtaagccgacgattttcatcggagaagaaatctctcacaaaaaacgcaccgaaaaagcagaaattgcggttccgtttttcggcaaaacacgcgcgactttttttctgttttggcgcgagttttttttcattctcgcgccaaaacgaaaaacttccggagccatgtttttttcgtttgtcgattctctattttctcgaaccggaagcatctctcgatatcaccttgcagagacaaaaaggcgaaactaaaaaaacgagcaatccgacatccacaaaaaaaataagtttcgttcgcccataaaaacgcctgcaaaaccccaaaaacacgtattaatacgtagagaaaatacgtgattctccgtataaaactgcgagtacagtagcataattttgcctttacaaagagagcagaaataggagacggatccaagaccgctgctttcttctgcattcacatggccacatgtaatgtaaacactgtaagaatttattacacaaaaagcatatgcagaatactaatccgacagctattatgaaaatgaaatatttccatcatccttcgggactacttccacgccttctcctgttattacttttaacgatgggggcggtagcatgtaccaaggaagataatccccatcatcatcatcatcatattagccaggcggtgcatgcggcgcatgcggaaattaacgaagcgggc

SEQ ID NO: 2 - DNA, 1257 bp, synthetic construct
  source 1..1257: mol_type = other DNA
gatcagcccacctcggacgaagtggcaacagtaaagatgtcgcttgacgatgtcgaaatgcgaggcggagacctctacagtggagaagatctgatcaagaaagtgcgcatattcgtctttcgtgaagggctaaacggtctttgggttctcgacaagcaaaaactatttgcttcggggcagtccgatttccaaaatcctttcacgatcagtgctcacgccggtcctcgccagatatacgtgatcgccaatgaacccgatgcccttacgacaaagctggataaaatccttttcaagaaagaacttgaggatatgcaagctcctgatgtgaacgagccgatagtccgaccattcaccatgacgggaatggctacggcgactcttaatccgcaaggcactgtgcaagcaaacatcagcctcaatcgtattgccgccaagatcacactggacatcaagcaagttactcccggcagcgatgtaatcaagattactaaggtgcagatccttcgcaacgccaagaactcgcgcctgctcgaaggcacaaacaaacctacaggatattggaattgggcgaatgcctgcgatcttccattgacgaacaacggcagcgcccagagcatcattcaggcatccgcgcctctctacgtatatgagaacataggatcagactcagactcttccggacgcgctacgcagcttgtggtggaagctctctacaatggtattaagacccgctactatgcctacgtgaacgataagaccactacggccaatcatcactactccattcgccgcaaccatcattacaaactggacgggacgatcacaaaaatgggcgaattcagcagcttgcttctcacgaccaccgtccttccgtggacagtagagaatttggattacggcttccttgtaccctatgtggcagaaataaatccgcatgcagtcattacgcaagacaacgtagttactttcgaaaactctttgtcgtttacggtcagaatcaaaggtcgagatggtagccgttggaaagcgactttagataatggattggaattcggttttgattccggcagtgctattgacggggctgccgatgggactaccgtctacactatcaaagtcaaagctttgaaacctaacggcatcggtatacaacgaagaacgaatctctttttcacggtagacggaaaaaaagtgattcttgacaagaatatcaacccccaacctaccgatattaagatcatacaacaaggactttaa